SEQUENCE LISTING (68 sequences)

SEQ ID NO: 1 - DNA, 20 bp, Artificial Sequence
  Synthetic Primer
gcctagaagtccggacaccg

SEQ ID NO: 2 - DNA, 20 bp, Artificial Sequence
  Synthetic Primer
gtggtgtggaacaaagcgaa

SEQ ID NO: 3 - DNA, 20 bp, Artificial Sequence
  Synthetic Primer
cctagaagtccggacaaccg

SEQ ID NO: 4 - DNA, 20 bp, Artificial Sequence
  Synthetic Primer
tggtgtggaacaaagccgaa

SEQ ID NO: 5 - DNA, 20 bp, Artificial Sequence
  Synthetic Primer
atgggattgtgaacagcgga

SEQ ID NO: 6 - DNA, 20 bp, Artificial Sequence
  Synthetic Primer
cgtgatttggactggcgatc

SEQ ID NO: 7 - DNA, 20 bp, Artificial Sequence
  Synthetic Primer
cagagcctcccaaacccgaa

SEQ ID NO: 8 - DNA, 20 bp, Artificial Sequence
  Synthetic Primer
tttgtggggagtgaccggtc

SEQ ID NO: 9 - DNA, 19 bp, Artificial Sequence
  Synthetic Primer
cagagcctcccaaaccgaa

SEQ ID NO: 10 - DNA, 20 bp, Artificial Sequence
  Synthetic Primer
aagacagtggagtgacgagg

SEQ ID NO: 11 - DNA, 20 bp, Artificial Sequence
  Synthetic Primer
aaagacctctagtgcacccc

SEQ ID NO: 12 - DNA, 20 bp, Artificial Sequence
  Synthetic Primer
agaagagaacgaagcgggaa

SEQ ID NO: 13 - DNA, 18 bp, Artificial Sequence
  Synthetic Primer
gagagggtggaggagggt

SEQ ID NO: 14 - DNA, 19 bp, Artificial Sequence
  Synthetic Primer
tgcattccaggctgagtga

SEQ ID NO: 15 - DNA, 20 bp, Artificial Sequence
  Synthetic Primer
ccccttggtactgtcctcac

SEQ ID NO: 16 - DNA, 20 bp, Artificial Sequence
  Synthetic Primer
aaagggtgcttgggacgtag

SEQ ID NO: 17 - DNA, 19 bp, Artificial Sequence
  Synthetic Primer
cctcactctccccacaagg

SEQ ID NO: 18 - DNA, 19 bp, Artificial Sequence
  Synthetic Primer
gcctgagtagccctggaaa

SEQ ID NO: 19 - DNA, 19 bp, Artificial Sequence
  Synthetic Primer
gcagctaaaggtcaaggcc

SEQ ID NO: 20 - DNA, 20 bp, Artificial Sequence
  Synthetic Primer
agctactcctcattcaagcc

SEQ ID NO: 21 - DNA, 23 bp, Artificial Sequence
  Synthetic Primer
aagtcctccctcaagctcatggc

SEQ ID NO: 22 - DNA, 18 bp, Artificial Sequence
  Synthetic Primer
attttcccggtggagagc

SEQ ID NO: 23 - DNA, 21 bp, Artificial Sequence
  Synthetic Primer
aggagaccagggcagaagatg

SEQ ID NO: 24 - DNA, 21 bp, Artificial Sequence
  Synthetic Primer
ctgggctttggtttggactcc

SEQ ID NO: 25 - DNA, 19 bp, Artificial Sequence
  Synthetic Primer
ccccatctgagtacgagcg

SEQ ID NO: 26 - DNA, 18 bp, Artificial Sequence
  Synthetic Primer
agccagttccacggtcag

SEQ ID NO: 27 - DNA, 25 bp, Artificial Sequence
  Synthetic Primer
agtgctggggaagatgatgacgacg

SEQ ID NO: 28 - DNA, 25 bp, Artificial Sequence
  Synthetic Primer
aggatggagacaatgaaacacgccc

SEQ ID NO: 29 - RNA, 16 nt, Artificial Sequence
  Synthetic Primer
cggggacaaggggaga

SEQ ID NO: 30 - DNA, 20 bp, Artificial Sequence
  Synthetic Primer
ccgcttaaacttgcctcgaa

SEQ ID NO: 31 - DNA, 20 bp, Artificial Sequence
  Synthetic Primer
aacaccccagatccacgaag

SEQ ID NO: 32 - DNA, 20 bp, Artificial Sequence
  Synthetic Primer
gcctctaaaagctccggatc

SEQ ID NO: 33 - DNA, 20 bp, Artificial Sequence
  Synthetic Primer
gtggacctgacctgccgtct

SEQ ID NO: 34 - DNA, 20 bp, Artificial Sequence
  Synthetic Primer
ggaggagtgggtgtcgctgt

SEQ ID NO: 35 - DNA, 20 bp, Artificial Sequence
  Synthetic Primer
gcctagaagtccggacaccg

SEQ ID NO: 36 - DNA, 20 bp, Artificial Sequence
  Synthetic Primer
gtggtgtggaacaaagcgaa

SEQ ID NO: 37 - DNA, 20 bp, Artificial Sequence
  Synthetic Primer
cctagaagtccggacaaccg

SEQ ID NO: 38 - DNA, 20 bp, Artificial Sequence
  Synthetic Primer
tggtgtggaacaaagccgaa

SEQ ID NO: 39 - DNA, 20 bp, Artificial Sequence
  Synthetic Primer
atgggattgtgaacagcgga

SEQ ID NO: 40 - DNA, 20 bp, Artificial Sequence
  Synthetic Primer
cgtgatttggactggcgatc

SEQ ID NO: 41 - DNA, 20 bp, Artificial Sequence
  Synthetic Primer
cagagcctcccaaacccgaa

SEQ ID NO: 42 - DNA, 20 bp, Artificial Sequence
  Synthetic Primer
tttgtggggagtgaccggtc

SEQ ID NO: 43 - DNA, 19 bp, Artificial Sequence
  Synthetic Primer
cagagcctcccaaaccgaa

SEQ ID NO: 44 - DNA, 20 bp, Artificial Sequence
  Synthetic Primer
aagacagtggagtgacgagg

SEQ ID NO: 45 - DNA, 20 bp, Artificial Sequence
  Synthetic Primer
aaagacctctagtgcacccc

SEQ ID NO: 46 - DNA, 20 bp, Artificial Sequence
  Synthetic Primer
agaagagaacgaagcgggaa

SEQ ID NO: 47 - DNA, 18 bp, Artificial Sequence
  Synthetic Primer
gagagggtggaggagggt

SEQ ID NO: 48 - DNA, 19 bp, Artificial Sequence
  Synthetic Primer
tgcattccaggctgagtga

SEQ ID NO: 49 - DNA, 20 bp, Artificial Sequence
  Synthetic Primer
ccccttggtactgtcctcac

SEQ ID NO: 50 - DNA, 20 bp, Artificial Sequence
  Synthetic Primer
aaagggtgcttgggacgtag

SEQ ID NO: 51 - DNA, 19 bp, Artificial Sequence
  Synthetic Primer
cctcactctccccacaagg

SEQ ID NO: 52 - DNA, 19 bp, Artificial Sequence
  Synthetic Primer
gcctgagtagccctggaaa

SEQ ID NO: 53 - DNA, 19 bp, Artificial Sequence
  Synthetic Primer
gcagctaaaggtcaaggcc

SEQ ID NO: 54 - DNA, 20 bp, Artificial Sequence
  Synthetic Primer
agctactcctcattcaagcc

SEQ ID NO: 55 - DNA, 23 bp, Artificial Sequence
  Synthetic Primer
aagtcctccctcaagctcatggc

SEQ ID NO: 56 - DNA, 18 bp, Artificial Sequence
  Synthetic Primer
attttcccggtggagagc

SEQ ID NO: 57 - DNA, 21 bp, Artificial Sequence
  Synthetic Primer
aggagaccagggcagaagatg

SEQ ID NO: 58 - DNA, 21 bp, Artificial Sequence
  Synthetic Primer
ctgggctttggtttggactcc

SEQ ID NO: 59 - DNA, 19 bp, Artificial Sequence
  Synthetic Primer
ccccatctgagtacgagcg

SEQ ID NO: 60 - DNA, 18 bp, Artificial Sequence
  Synthetic Primer
agccagttccacggtcag

SEQ ID NO: 61 - DNA, 25 bp, Artificial Sequence
  Synthetic Primer
agtgctggggaagatgatgacgacg

SEQ ID NO: 62 - DNA, 25 bp, Artificial Sequence
  Synthetic Primer
aggatggagacaatgaaacacgccc

SEQ ID NO: 63 - DNA, 20 bp, Artificial Sequence
  Synthetic Primer
cgtgtggacaaggtggatga

SEQ ID NO: 64 - DNA, 20 bp, Artificial Sequence
  Synthetic Primer
ccgcttaaacttgcctcgaa

SEQ ID NO: 65 - DNA, 20 bp, Artificial Sequence
  Synthetic Primer
aacaccccagatccacgaag

SEQ ID NO: 66 - DNA, 20 bp, Artificial Sequence
  Synthetic Primer
gcctctaaaagctccggatc

SEQ ID NO: 67 - DNA, 20 bp, Artificial Sequence
  Synthetic Primer
gtggacctgacctgccgtct

SEQ ID NO: 68 - DNA, 20 bp, Artificial Sequence
  Synthetic Primer
ggaggagtgggtgtcgctgt

(19) United States
(12) Patent Application Publication
(10) Pub. No.: US 2024/0026320 A1
(43) Pub. Date: Jan. 25, 2024

(54) MATERIALS AND METHODS FOR MODIFYING EXPRESSION OF MYOSIN HEAVY CHAIN GENES

(71) Applicant: NORTHWESTTERN UNIVERSITY, Evanston, IL (US)

(72) Inventors: Elizabeth M. McNally, Oak Park, IL (US); Anthony Martin Gacita, Chicago, IL (US)

(21) Appl. No.: 18/265,135

(22) PCT Filed: Dec. 3, 2021
(86) PCT No.: PCT/US21/61750

§ 371 Date: Jun. 2, 2023

Related U.S. Application Data
(60) Provisional application No. 63/121,560, filed on Dec. 4, 2020.

(51) Int. Cl. C12N 9/22; C12N 15/86; C12N 15/10; A61P 9/00

(52) U.S. Cl. CPC C12N 9/22; C12N 15/86; C12N 15/102; A61P 9/00; C12N 2750/14143; C12N 2310/20

(57) ABSTRACT

Described herein is a method for editing the MHY7 gene in a cell by genome editing comprising introducing into the cell one or more deoxyribonucleic acid (DNA) endonucleases to effect one or more double stranded breaks (DSBs) within or near enhancer regions of the MYH7 gene or MYH6 gene that results in deletion of one or more enhancer regions of the MYH7 gene.

### CROSS-REFERENCE TO RELATED APPLICATIONS

This application claims the priority benefit under 35 U.S.C. § 119(e) of U.S. Provisional Patent Application No. 63/121,560, filed Dec. 4, 2020, which is incorporated herein by reference in its entirety.

### INCORPORATION BY REFERENCE OF INFORMATION SUBMITTED ELECTRONICALLY

This application contains, as a separate part of the disclosure, a Sequence Listing in computer readable form (Filename: 2020-154_Seqlisting.txt; Size: 13,124 bytes; Created: Dec. 2, 2021), which is incorporated by reference in its entirety.

### FIELD OF THE INVENTION

The disclosure is directed to the use of genome editing to both modify expression of myosin heavy chain (MHC) genes associated with cardiomyopathy.

### BACKGROUND

Cardiomyopathies are heart muscle disorders which represent a heterogeneous group of diseases that often lead to progressive heart failure with significant morbidity and mortality. Common symptoms include dyspnea, exercise and activity intolerance and peripheral oedema, and risks of having dangerous forms of irregular heart rate and sudden cardiac death are increased. The most common form of cardiomyopathy is dilated cardiomyopathy. Dilated cardiomyopathy is a heart muscle disorder characterized by dilatation and systolic dysfunction of the left or both ventricles (Elliott, P., Andersson, B., Arbustini. E., Bilinska, Z., Cecchi, F., Charron, P., Dubourg, O., Ktihl, U., Maisch, B., McKenna, W. J., et al. (2008) Classification of the cardiomyopathies: a position statement from the European society of cardiology working group on myocardial and pericardial diseases. Eur. Heart J., 29, 270-276). The ventricular walls become thin and stretched, compromising cardiac contractility and ultimately resulting in poor left ventricular function. Other forms of cardiomyopathy include hypertrophic, arrhythmogenic and restrictive. Chronic or acute coronary artery disease can also lead to ischemic cardiomyopathy and cause heart failure.

Protein coding mutations in over 100 genes have been linked to autosomal dominant cardiomyopathy, which leads to heart failure and significant burden¹⁻³. A well-recognized clinical feature of genetic cardiomyopathy is its variable phenotypic expression. Genetic cardiomyopathy demonstrates an age-dependent penetrance, variable expressivity, and variable clinical presentations, even in patients sharing identical primary mutations^{4,5}. Protein coding variants have been described as altering the phenotypic expression of primary cardiomyopathy-causing mutations⁵⁻⁷. However, the contribution of noncoding variation as modifiers of the clinical presentation of cardiomyopathy has been less well investigated.

Noncoding regions of the genome harbor important regulatory sequences that control the expression of genes through both distal enhancers and proximal gene promoters⁸. ChIP-seq, ATAC-seq, and CAGE-seq can mark genomic regions as having regulatory function, but do not provide information on their gene target. Chromatin conformation assays evaluate genomic three-dimensional organization and link enhancers to their target genes. However, as enhancer function is dependent on tissue-specific transcription factors, assays for enhancer function or targets require the context of relevant tissues/cells.

### SUMMARY

In one aspect, described herein is a method for editing the myosin heavy chain 7 (MHY7) gene in a cell comprising introducing into the cell one or more deoxyribonucleic acid (DNA) endonucleases to induce one or more double stranded breaks (DSBs) within chr14:23870150-23924866 as designated in the human genome browser, build 38 (hg38), of the MYH7 gene that results in deletion of an enhancer region of the MYH7 gene. In some embodiments, the method results in decreased MYH7 expression and increased MYH6 expression in the cell, relative to a cell into which the DNA endonuclease was not introduced.

In some embodiments, the enhancer region is upstream (e.g., within 500 bp) of the MYH6 gene. In some embodiments, the enhancer region is within the MYH6 gene. In some embodiments, the enhancer region is downstream (e.g., within 500 bp) of the MYH7 gene. In some embodiments, the enhancer region is MYH7-C6, MYH7-C3, MYH7-C4 or MYH7-C5. In some embodiments, the enhancer region MYH7-C3 is deleted from the MYH7 gene.

In some embodiments, the one or more DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog thereof.

In some embodiments, the method comprises introducing into the cell one or more polynucleotides encoding the one or more DNA endonucleases.

In some embodiments, the method comprises introducing into the cell one or more ribonucleic acids (RNAs) encoding the one or more DNA endonucleases.

In some embodiments, the method further comprises introducing into the cell one or more guide ribonucleic acids (gRNAs). In some embodiments, the one or more guide RNAs (gRNAs) comprise a nucleotide sequence set forth in SEQ ID NOs: 1-68.

In some embodiments, the one or more DNA endonucleases is pre-complexed with one or more gRNAs. In some embodiments, the DNA endonuclease and one or more guide RNAs are delivered by a viral vector. Exemplary viral vectors include, but are not limited to, a herpes virus vector, an adeno-associated virus (AAV) vector, an adeno virus vector, or a lentiviral vector. In some embodiments, the viral vector is an adeno-associated virus (AAV) vector. In some embodiments, the AAV vector is recombinant AAV5, AAV6, AAV8, AAV9, or AAV7.

In another aspect, described herein is a method of improving heart function in a subject suffering from cardiomyopathy comprising administering to the subject an agent that both increases myosin heavy chain 6 (MYH6) gene expression and decreases myosin heavy chain 7 (MYH7) gene expression in a cardiac cell of the subject. In some embodiments, the agent is one or more deoxyribonucleic acid (DNA) endonucleases to effect one or more double stranded breaks (DSBs) within or near enhancer regions of the MYH7 gene of the MYH6 gene that results in deletion of one or more enhancer regions of the MYH7 gene.

In another aspect, described herein is a method for editing the LMNA gene in a cell by genome editing comprising introducing into the cell one or more deoxyribonucleic acid (DNA) endonucleases to effect one or more double stranded breaks (DSBs) within or near chr1:155937201-156100640 as designated in the human genome browser, build 38 (hg38) of the LMNA gene that results in deletion of one or more enhancer regions of the LMNA gene. In some embodiments, the one or more enhancer regions is LMNA-C1, LMNA-C2, LMNA-C3, LMNA-C4, LMNA-C5, or LMNA-C6.

In another aspect, described herein is a composition comprising one or more guide RNAs (gRNAs) comprise a nucleotide sequence set forth in SEQ ID NOs: 1-68 and a pharmaceutically acceptable carrier, diluent or adjuvant.

### BRIEF DESCRIPTION OF THE FIGURES

FIG. 1. Integrated epigenomic analysis identifies candidate regulatory regions for MYH7 and LMNA. MYH7 encodes α-myosin heavy chain (MHC), the major contractile protein in the human left ventricle; mutations in MYH7 are a leading cause of inherited cardiomyopathy. Mutations in LMNA, which encodes lamin A/C also contribute to inherited cardiomyopathies. We intersected enhancer data from multiple sources to identify regulatory regions around these genes. A. The MYH6/7 genes are in near two clusters of candidate enhancers, highlighted in yellow boxes, which may regulate their expression. B. Integrated epigenomic analysis identified three candidate enhancer clusters at the LMNA locus. The labels on the left indicate the data and cell/tissue source (full source listing is found in Table 2). pcHi-C, promoter capture Hi-C. LV, left ventricle. iPSC-CMs, iPSC-derived cardiomyocytes.

FIG. 2. Enhancer activity in IPSC-Derived Cardiomyocytes (iPSC-CMs). A luciferase reporter assay was used to test for enhancer activity in iPSC-CMs. The position of candidate enhancers is shown along the top in colored boxes. The clusters in FIG. 1 were evaluated as smaller regions. A. Regions from 4 of 5 candidate enhancer regions demonstrated activity in iPSC-CMs, with the highest activity for MYH7 C3. B. Five of six candidate enhancer regions for LMNA showed activity in iPSC-CMs, with the highest being LMNA C5. These data indicate that the candidate MYH7 and LMNA enhancers represent true enhancer regions in a cardiac system. Data is displayed as fold change to negative control 500 bp genomic desert region with mean±SD. Significance vs negative control determined by nonparametric one-way ANOVA. *<0.03, **<0.0021, ***<0.0002, ****<0.0001.

FIG. 3. Deletion of the MYH7 C3 enhancer increases MYH6 and reduces MYH7 mRNA and protein and produces hyperdynamic function in engineered heart tissues. A. Gene editing was used to delete the MYH7 C3 enhancer heterozygously (+/−) or homozygously (−/−). MYH6 and MYH7 mRNA expression was assayed by qPCR and showed a dose-dependent increase in MYH6 expression and reduction in MYH7 expression. Therefore, the MYH7 C3 enhancer is required for MYH7 expression. B. Deletion of the MYH7 C4 enhancer had little effect, demonstrating a specificity of these findings to MYH7 C3. C. α-MHC and β-MHC protein ratios were quantified using SDS-PAGE. D. Quantification of α-MHC/β-MHC protein ratios in C and match the differences seen at the RNA level E. Representative images of engineered heart tissues (EHTs) containing unedited or MYH7 C3 homozygous deleted iPSC-CMs. F. Average time to peak measurements of EHT contractions containing unedited or MYH7 C3 deleted cells showed an increase in time to peak in MYH7 C3 deleted EHTs, consistent with the shift from MYH7/β-MHC to MYH6/α-MHC and the known faster ATPase cycle for α-MHC. Each point represents the average time to peak measurement of a single EHT across multiple contractions. All data shown as mean±SD. * determined by one-way ANOVA. *<0.03, **<0.0021, ***<0.0002, ****<0.0001.

FIG. 4. CRISPR-Cas9 enhancer deletion strategy successfully removes MYH7 enhancer regions. A. Schematic of CRISPR-Cas9 deletion strategy and PCR primers used for genotyping. B. Agarose gels of 3-primer PCRs on genomic DNA from IPSCs treated with guides targeting MYH7 candidate enhancers 3 and 4 demonstrating successful deletion. C. Top, schematic representation of the location of the MYH6/7 regulatory variant. Bottom, agarose gel of 3-primer PCR of genomic DNA from IPSCs treated with guides targeting the region overlapping the MYH6/7 regulatory variant showing successful deletion.

FIG. 5. Genomic variation in MYH7 enhancer regions. A. We queried MYH7 enhancers for naturally occurring sequence variants for those that overlapped cardiac transcription factor binding motifs, and/or were correlated with MYH7 expression in the GTEx eQTL dataset. rs7403916 and rs373958405 fall within MYH7 C2 and disrupt NKX2.5 motifs. These variants were evaluated for reporter activity in iPSC-CMs and rs373958405 demonstrates reduced activity compared to the reference allele, which indicates that this variant may reduce MYH6/7 expression by disrupting the enhancer activity of MYH7 C2. B. MYH7 C3 contains rs7149564 and chr14_23912371_C. rs7149564 disrupts an NKX2.5 motif and results in a trending reduction in iPSC-CM luciferase signal. chr14_23912371_C generates a TCF21 motif and results in an increased iPSC-CM luciferase signal. MYH7 C4 contains rs116554832 and rs10873105. rs116554832 disrupts a TBX5 motif and results in a reduced iPSC-CM luciferase signal. rs10873105 is correlated with MYH7 expression in GTEx skeletal muscle data and creates a Hox10 motif. This variant results in an increased iPSC-CM luciferase signal. These data indicate that sequence variants that overlap transcription factor binding sites within enhancer regions can alter enhancer function and may affect MYH7 gene expression. The ChIP-seq and homer datasets are listed in Table 2. All data shown as mean±SD. Significance determined by unpaired t-test. *<0.03, **<0.0021, ***<0.0002, ****<0.0001.

FIG. 6. Computational pipeline to identify enhancer modifying variants. A. Schematic of pipeline filtering steps to identify enhancer modifying variants (EMVs) that are within enhancer regions and transcription factor binding sites. All datasets used were generated in iPSC-CMs (see Table 2) B. This strategy disproportionately identified significant GTEx eQTLs from heart tissues versus non-heart tissues, and disproportionately identified rare alleles, indicating tissue specificity and sequence conservation (C). Significance determined in B & C by Fisher's exact test. D. Luciferase reporter assay in iPSC-CMs for selected regions containing variants of interest identified through this analysis indicates that 4/5 variants overlapped enhancer regions. Significance vs negative control was determined by nonparametric one-way ANOVA. E. Luciferase signal for reference and alternative alleles of selected variants identified through this pipeline. Variants that altered enhancer function (EMVs) are highlighted in yellow. These variants are well positioned to alter cardiac gene expression of important genes. Significance determined by unpaired t-test. All data shown as mean±SD. *<0.03, **<0.0021, ***<0.0002, ****<0.0001.

FIG. 7. Deletion of the C6 enhancer region alters MYH6/7 expression. A. Schematic demonstrating the location of the MYH6/7 C6 enhancer region, which indicates that the rs875908 EMV closer to the MYH7 transcriptional start site than previously tested MYH7 enhancers. B. iPSC-CM MYH6 and MYH7 expression levels in cells deleted heterozygously or homozygously for the C6 enhancer region containing rs875908. MYH6/7 levels were assayed by qPCR, and show a dose-dependent reduction in MYH7. Therefore, rs875908, is within an enhancer regions required for strong MYH7 expression in cardia cells C. SDS-PAGE analysis of myosin heavy chain protein isoforms in MYH7 C6−/+ and −/−cells. D. Quantification of α-MHC/β-MHC ratios in C indicating that RNA differences are present at the protein level. Significance determined by one-way ANOVA. *<0.03, **<0.0021, ***<0.0002, ****<0.0001.

FIG. 8. Correlation of MYH6/7 rs875908 EMV with MYH7 mRNA cardiac expression and longitudinal shift in left ventricular dimensions over time. A. UCSC genome browser screenshot showing the location of the MYH6/7 regulatory variant and that it is bound by both GATA4 and TBX5 in cardiac cells. The variant disrupts a site within the TBX5 transcription factor motif. B. eQTL data from the GTEx project indicating that the variant genotype correlates with MYH7 expression in three muscle tissues. C. Association of variant status with LVIDd/BSA over time in cardiomyopathy cases from NU genomes cohort. D. Association of variant genotype with LVPWd/BSA over time in in cardiomyopathy cases from NU genomes cohort. These data indicate that rs875908 modifies changes of left ventricular morphology over time in patients with cardiomyopathy. Significance determined using a linear regression model corrected for genetic ancestry and sex. LVIDd/BSA, left ventricular internal diameter during diastole corrected for body surface area. LVPWd/BSA, left ventricular posterior wall thickness during diastole corrected for body surface area.

FIG. 9. Schematic diagram of the MYH6/7 locus during cardiac development. At the uninduced locus, the MYH7 and MYH6 promoter regions form a complex with a super enhancer containing the MYH7 C3 enhancer (the effect of the super enhancer is depicted as the yellow hue). In early development, this super enhancer is primarily in contact with the MYH6 promoter, which recruits cardiac transcription factors, chromatin remodelers, and transcription machinery to drive MYH6 expression. During development, activation of the MYH7 C3 region reorganizes the complex and the MYH7 promoter preferentially contacts the regulatory regions. Through competition for transcriptional machinery or through an independent separate mechanism, the MYH6 gene is downregulated. Cells that lack the MYH7 C3 region are able to set up the chromatin structure of the locus but are unable to switch interactions to the MYH7 promoter, causing an upregulation of MYH6 and a downregulation of MYH7. This model highlights the importance of an MYH7 enhancer which is an attractive therapeutic target. TFs, transcription factors

FIG. 10. Negative control region reporter assay activity in HL-1 cells and IPSC-CMs. HL-1 cells are a mouse atrial cell line 18. Expression of MYH6/7 differs between atrial and ventricles and between mouse and human ventricles 19. A. Luciferase assay using HL-1 cells including multiple negative control regions. Average n=18 across three different days. B. Luciferase assay data for multiple negative control regions in IPSC-CMs. Average n=16 across two differentiations. Desert represents genomic regions with little or no evidence of enhancer function in left ventricle tissue. Scrambled represents randomly selected nucleotides. Significance vs desert 500 bp determined by nonparametric one-way ANOVA with Dunn's multiple comparisons correction. *<0.03, **<0.0021, ***<0.0002, ****<0.0001.

FIG. 11. Reporter assay for candidate enhancer regions of MYH7 and LMNA in HL-1 cells. A. Above, color-coded schematic of candidate MYH7 enhancers identified in FIG. 1. Below, data from luciferase reporter assay in HL-1s for full and partial candidate enhancer regions. B. Above, color-coded schematic of candidate LMNA enhancers identified in FIG. 1. Below, data from luciferase reporter assay in HL-1s for full and partial candidate enhancer regions. Data displayed as fold change to negative control genomic 500 bp desert region with mean+/−SD. Average n=17 across three separate days. Significance vs negative control determined by nonparametric one-way ANOVA with Dunn's multiple comparisons correction. *<0.03, **<0.0021, ***<0.0002, ****<0.0001.

FIG. 12. Validation of gene edited iPSCs and IPSC-CMs. Nonhomologous end joining CRISPr-Cas9 was used to generate guided deletions in iPSCs. Resulting clones were treated isolated analyzed for common chromosomal rearrangements. A. Results from the hPSC genetic analysis test kit (Stem Cell Technologies) assaying common chromosomal rearrangements in CRISPr treated IPSCs. B. IPSC-CM purity measurements evaluating the percent cardiac troponin T (cTNT) cells across different enhancer deletion lines. cTnT, cardiac troponin T. No significant differences were found between unedited and CRISPr treated cells by one-way ANOVA.

FIG. 13. Phenotypic regressions using the NU genomes cohort. A. Association of variant status with LVIDd/BSA over time in the NU genomes cohort (n=387). B. Association of variant genotype with LVPWd/BSA overtime in the NU genomes cohort. Significance determined using a linear regression model corrected for race and sex. LVIDd/BSA, left ventricular internal diameter during diastole corrected for body surface area. LVPWd/BSA, left ventricular posterior wall thickness during diastole corrected for body surface area.

FIG. 14. Deletion of the MYH7-C3 and MYH7-C6 enhancer affects MYH7 and MYH6 expression levels across multiple clones. A. Reduction of MYH7 mRNA expression in MYH7-C3-deleted cells was observed across multiple independent clones. B. Increase of MYH6 expression in MYH7-C3-deleted cells was observed across multiple independent clones. C. Reduction of MYH7 mRNA expression in MYH7-C6-deleted cells was observed across multiple clones. D. Changes in MYH6 mRNA expression in MYH7-C6-deleted cells are consistent across multiple independent clones. All data shown as mean±SD. * determined by one-way ANOVA with Dunnett's multiple comparisons test. **<0.0021, ****<0.0001.

FIG. 15. Regulatory Variant VISTA Overlap and myosin heavy chain RNA and protein level correlations. A. Overlap between variants identified by our pipeline and the enhancer regions tested in the VISTA database. B. Regression between the MYH6/MYH7 ratio determined by qPCR and the α/β MHC ratio determined by SDS-PAGE. Significance determined by linear regression. ****<0.0001.

FIG. 16. Deletion of the MYH7-C3 enhancer produces hyperdynamic function in engineered heart tissues. A. Average contraction amplitude measurements of EHT contractions containing unedited or MYH7-C3 deleted cells showed a decrease in contraction amplitude in MYH7-C3 deleted EHTs. Each point represents the average time to peak measurement of a single EHT across multiple contractions (unedited n=14, MYH7 C3+/− n=3, MYH7 C3−/− n=7.) All data shown as mean±SD. * determined by one-way ANOVA with Dunnett's multiple comparisons correction. *<0.03, **<0.0021, ***<0.0002, ****<0.0001.

FIG. 17. Full map of putative MYH7 Enhancers.

FIG. 18. Full map of putative LMNA Enhancers.

FIG. 19. Full map of MICAL2 Regulatory Variant.

FIG. 20. Full map of MYH6/7 Regulatory Variant.

FIG. 21. Full map of NPPA Regulatory Variant.

FIG. 22. Full map of TNNT2 Regulatory Variant.

FIG. 23. Full map of GATA4 Regulatory Variant.

### DETAILED DESCRIPTION

Inherited cardiomyopathy associates with a range of phenotypic expression. As described in the Examples, epigenomic profiling from multiple sources was superimposed, including promoter-capture chromatin conformation information, to identify candidate enhancer regions for two cardiomyopathy genes, MYH7 and LMNA. Enhancer function was validated in human cardiomyocytes derived from induced pluripotent stem cells and revealed enhancer regions implicated the switch of MYH6 and MYH7 expression. By querying human genomic variation, multiple sequence changes were identified that modified enhancer function by creating or interrupting transcription factor binding sites. rs875908, which is 2 KB 5′ of MYH7, associated with longitudinal clinical features of cardiomyopathy in a biobank with clinical imaging and genetic data.

Myosin Heavy Chain Genes, MYH7 and MYH6

Mutations in MYH7 are a common cause of hypertrophic cardiomyopathy while mutations in LMNA are a common cause of dilated cardiomyopathy with arrhythmias^{4,9}. MYH7 encodes β-myosin heavy chain (MHC), which is the major left ventricular myosin heavy chain in the adult human.

In humans, both MYH7 (α-MHC) and MYH6 (β-MHC) are expressed in myocardium and cause cardiomyopathy when mutated (Carniel et al., Circulation 112:-54-59, 2005; Kamisago et al., NEJM, 343:1688-1696, 2000). These genes are in tandem on chromosome 14, with MYH6 located 5.3 kb downstream of MYH7, and their expression is developmentally regulated. MYH6 is mainly expressed in embryonic heart, whereas MYH7 becomes the predominant adult isoform (Lowes et al., J. Clin. Invest., 100:2315-2324, 1997).

An integrative analysis was used that relied on >20 publicly-available heart enhancer function and enhancer target datasets to identify MYH7 and LMNA left ventricle enhancer regions. The activity of these regions was confirmed using reporter assays and CRISPR-mediated deletion in human cardiomyocytes derived from induced pluripotent stem cells (iPSC-CMs). These regulatory regions contained sequence variants within transcription factor binding sites that altered enhancer function. Extending this strategy genome-wide, an enhancer modifying variant was identified upstream of MYH7. This common variant correlated with MYH7 expression in the GTEx eQTL dataset. Finally, the variant was also determined to be correlated with a more dilated left ventricle over time. These findings link noncoding enhancer variation to cardiomyopathy phenotypes and provide direct evidence of the importance of genetic background.

In one aspect, described herein is a method for editing the MHY7 gene in a cell comprising introducing into the cell one or more deoxyribonucleic acid (DNA) endonucleases to induce one or more double stranded breaks (DSBs) within or near chr14:23870150-23924866 as designated in the human genome browser, build 38 (hg38), that results in deletion of an enhancer region of the MYH7 gene. In some embodiments, the enhancer region is upstream (e.g., within 500 bps) of the MYH6 gene. In some embodiments, the enhancer region is upstream within 10 bp, 20 bp, 30 bp, 40 bp, 50 bp, 60 bp, 70 bp, 80 bp, 90 bp, 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp or 500 bp of the MYH6 gene. In some embodiments, the enhancer region is MYH7-C1 or MYH7-C2. In some embodiments, the enhancer region is downstream of the MYH7 gene (e.g., within 500 bps). In some embodiments, the enhancer region is downstream within 10 bp, 20 bp, 30 bp, 40 bp, 50 bp, 60 bp, 70 bp, 80 bp, 90 bp, 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp or 500 bp of the MYH7 gene. In some embodiments, the enhancer region is MYH7-C6, MYH7-C3, MYH7-C5 or MYH7-C4. In some embodiments, the enhancer region MYH7-C3 is deleted from the MYH7 gene. Locations of the various enhancer regions of the MYH7 gene are provided below in Table A.

TABLE A

| Region | Size | | Distance |
|---|---|---|---|
| Name | (bp) | Coordinates (hg19) | to TSS (bp) |
| MYH7-C1-2 | 673 | chr14:23870150-23870823 | 34,047 |
| MYH7-C1-3 | 698 | chr14:23870761-23871458 | 33,412 |
| MYH7-C1-4 | 697 | chr14:23871436-23872136 | 32,734 |
| MYH7-C2 | 2072 | chr14:23876121-23878188 | 26,682 |
| MYH7-C2-1 | 694 | chr14:23877446-23878141 | 26,729 |
| MYH7-C2-2 | 838 | chr14:23876221-23877058 | 27,812 |
| MYH7-C2-3 | 844 | chr14:23876782-23877626 | 27,244 |
| MYH7-C3 | 961 | chr14:23912000-23912961 | −7,130 |
| MYH7-C4-1 | 1400 | chr14:23913940-23915344 | −9,070 |
| MYH7-C4-2 | 2209 | chr14:23915187-23917391 | −10,317 |
| MYH7-C5 | 2223 | chr14:23922666-23924886 | −17,796 |
| MYH7-C5-1 | 790 | chr14:23923381-23924171 | −18,511 |

In some embodiments, the methods result in decreased MYH7 expression and increased MYH6 expression in the cell, compared to a cell that does not comprise the endonuclease.

LMNA Gene

The LMNA gene encodes nuclear lamin A and nuclear lamin C, intermediate filament proteins that are components of the nuclear lamina. Most disease-causing LMNA mutations affect the heart, causing a dilated cardiomyopathy, with or without skeletal muscle involvement (Lu et al., Disease Models and Mechanisms, 4:562-568, 2011).

In another aspect, described herein is a method for editing the LMNA gene in a cell by genome editing comprising introducing into the cell one or more deoxyribonucleic acid (DNA) endonucleases to effect one or more double stranded breaks (DSBs) within or near chr1:155937201-156100640 as designated in the human genome browser, build 38 (hg38) of the LMNA gene that results in deletion of one or more enhancer regions of the LMNA gene. In some embodiments, the one or more enhancer regions is LMNA-C1, LMNA-C2, LMNA-C3, LMNA-C4, LMNA-C5, or LMNA-C6. Locations of the various enhancer regions of the LMNA gene are provided below in Table B.

TABLE B

| Region | Size | | Distance |
|---|---|---|---|
| Name | (bp) | Coordinates (hg19) | to TSS (bp) |
| LMNA-C1-1 | 1156 | chr1:155937201-155938359 | −146,102 |
| LMNA-C1-2 | 1210 | chr1:155936009-155937220 | −147,241 |
| LMNA-C2 | 928 | chr1:23904382-23905597 | −17,195 |
| LMNA-C3-1 | 1108 | chr1:156074366-156075480 | −8,981 |
| LMNA-C3-2 | 1199 | chr1:156073216-156074415 | −10,046 |
| LMNA-C4-1 | 1211 | chr1:156092084-156093294 | 7,623 |
| LMNA-C4-2 | 1392 | chr1:156093103-156094494 | 8,642 |
| LMNA-C5 | 850 | chr1:156095724-156096574 | 11,263 |
| LMNA-C6 | 1101 | chr1:156099538-156100640 | 15,077 |

CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the locus, biogenesis of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” The repeats can form hairpin structures and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures. In some embodiments, the DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog thereof.

Examples of various Cas9 proteins and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM facilitates binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and International Patent Application Publication Number WO2013/176772 (incorporated herein by reference) provides numerous examples and applications of the CRISPR/Cas endonuclease system for site-specific gene editing.

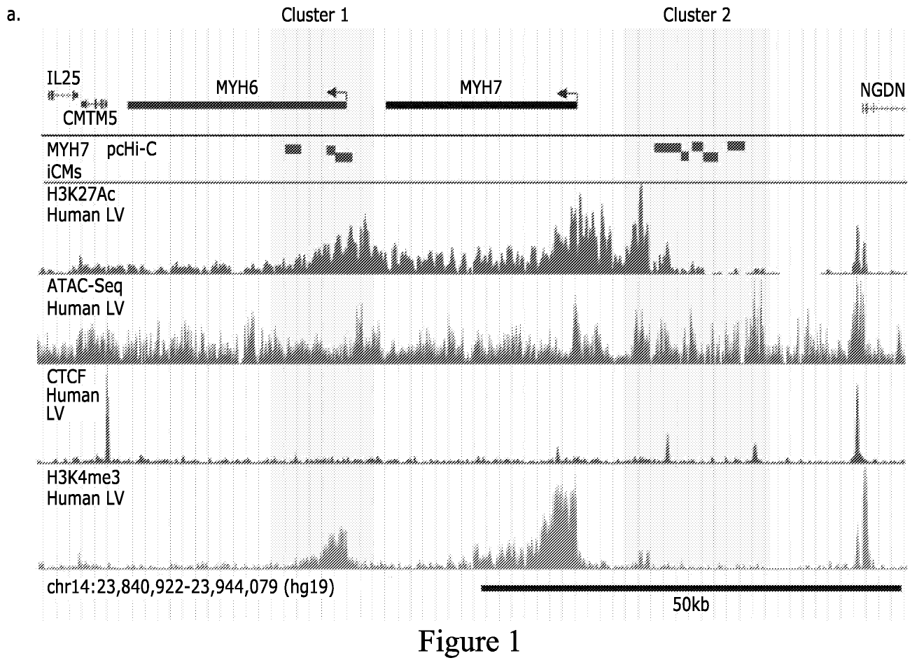

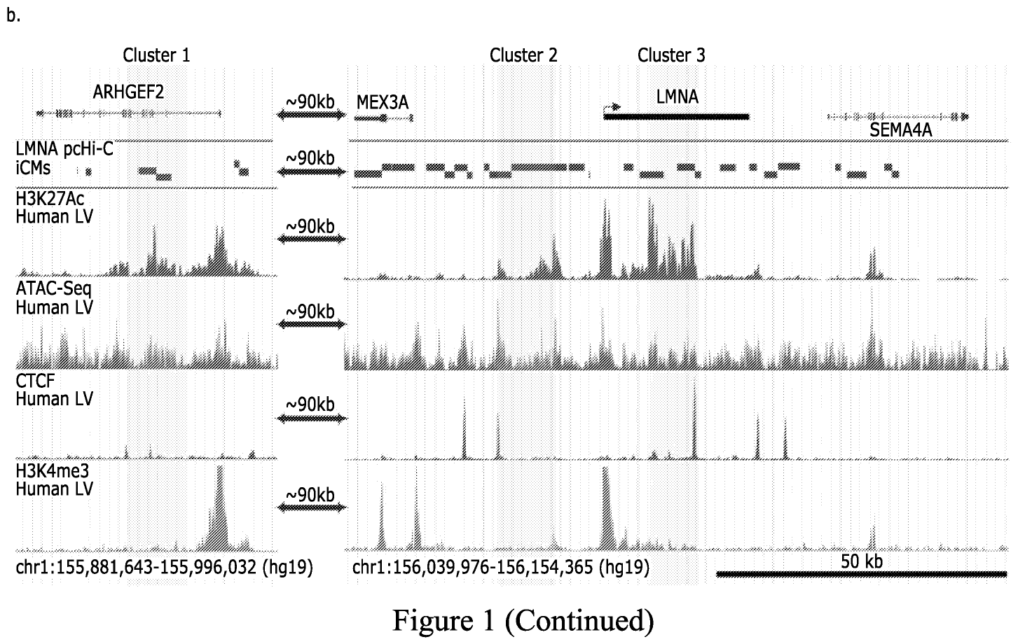

Exemplary CRISPR/Cas polypeptides include the Cas9 polypeptides in FIG. 1 of Fonfara et al., Nucleic Acids Research, 42: 2577-2590 (2014) (incorporated herein by reference). The CRISPR/Cas gene naming system has undergone extensive rewriting since the Cas genes were discovered.

Cas9 polypeptides can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. HDR can occur when a homologous repair template, or exogenous nucleic acid, is available.

In some embodiments, the DNA endonuclease is introduced to the cell as a protein (i.e., a protein-based system). Typically, the cell is treated chemically, electrically, or mechanically to allow Cas9 nuclease entry into the cell. Alternatively, in some embodiments, the endonuclease is introduced to the cell as a nucleic acid (e.g., DNA or mRNA) under conditions which allow production of the nuclease. Guide RNA also is introduced into the cell.

In some embodiments, the methods described herein comprise introducing one or more guide RNAs into the cell. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a tracrRNA sequence. In the Type II guide RNA, the CRISPR repeat sequence and tracrRNA sequence hybridize to each other to form a duplex. The duplex binds a site-directed polypeptide, such that the guide RNA and site-direct polypeptide form a complex. The guide RNA provides target specificity to the complex by virtue of its association with the Cas9 nuclease. The guide RNA thus directs the activity of the Cas9 nuclease.

Exemplary gRNA for use in the methods described herein include, but are not limited to, the gRNAs provided in Table 1 in Example 1.

In some embodiments, the methods described herein comprise delivering the endonuclease and one or more gRNAs to the cell by a viral vector. Any of the expression vectors described herein may be used to deliver endonuclease-encoding nucleic acid into the cell; in many aspects, the expression vector is a plasmid. Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In some embodiments, the viral vector is AAV2, AAV3, AAV3B, AAV5, AAV6, AAV6.2, AAV7, AAVrh.64R1, AAVhu.37, AAVrh.8, AAVrh.32.33, AAV8, AAV9, AAVrh10, or AAVLK03. In other embodiments, the viral vector may a lentivirus vector.

In some embodiments, a viral vector comprises one or more transcription and/or translation control elements. Depending on the host/vector system utilized, any of a number of suitable transcription and translation control elements, including constitutive and inducible promoters, transcription enhancer elements, transcription terminators, etc., may be used. In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

The Cas9 nuclease-encoding nucleic acid is operably linked to a promoter that drives protein expression. For expressing small RNAs, including guide RNAs used in connection with Cas or Cpf1 endonuclease, promoters such as RNA polymerase III promoters, including for example U6 and H1, can be advantageous. Suitable promoters, as well as parameters for enhancing the use of such promoters, are known in art, and additional information and approaches are regularly being described; see, e.g., Ma, H. et al., Molecular Therapy—Nucleic Acids 3, e161 (2014) doi:10.1038/mtna.2014.12.

In some embodiments, the nucleotide sequence encoding the guide RNA may be located on the same vector comprising the nucleotide sequence encoding the endonuclease. In some embodiments, expression of the guide RNA and of the endonuclease may be driven by their own corresponding promoters. In some embodiments, expression of the guide RNA may be driven by the same promoter that drives expression of the endonuclease. In some embodiments, the guide RNA and the endonuclease transcript may be contained within a single transcript. For example, the guide RNA may be within an untranslated region (UTR) of the endonuclease transcript. In some embodiments, the guide RNA may be within the 5′ UTR of the transcript. In other embodiments, the guide RNA may be within the 3′ UTR of the transcript.

Treatment Methods

The Examples provided herein demonstrate that deletion of the MYH7-C3 enhancer region reduced MYH7 expression in iPSC-CMs, and, correspondingly, deletion of the MYH7-C3 enhancer increased MYH6 expression, resulting in an αMHC/βMHC ratio that increased heart function in engineered heart tissues. Thus, in another aspect, the disclosure provides a method for increasing heart function in a subject in need thereof comprising administering to the subject an agent that increases MYH6 and decreases MYH7 gene expression in a cell of the subject. In some embodiments, the subject is suffering from cardiomyopathy, heart failure, arrhythmia, ischemic heart disease, non-ischemic heart disease and exercise or activity intolerance.

As used herein, “cardiomyopathy” refers to any disease or dysfunction of the myocardium (heart muscle) in which the heart is abnormally enlarged, thickened and/or stiffened. As a result, the heart muscle's ability to pump blood is usually weakened and unable to meet the demands of the body, often leading to congestive heart failure. The disease or disorder can be, for example, inflammatory, metabolic, toxic, infiltrative, fibrotic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. The cardiac disorder may be pediatric in origin. Cardiomyopathy includes, but is not limited to, cardiomyopathy (dilated, hypertrophic, restrictive, arrhythmogenic, ischemic, genetic, idiopathic and unclassified cardiomyopathy), sporadic dilated cardiomyopathy, X-linked Dilated Cardiomyopathy (XLDC), acute and chronic heart failure, right heart failure, left heart failure, biventricular heart failure, congenital heart defects, myocardiac fibrosis, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, systolic heart failure, diabetic heart failure and accumulation diseases. In some embodiments, the heart failure includes reduced ejection fraction. In further embodiments, the heart failure includes preserved ejection fraction.

In some embodiments, the methods described herein treat the cardiomyopathy in the subject. It will be appreciated that “treating cardiomyopathy” does not require complete amelioration of the disorder; “treating” includes any improvement in a symptom or manifestation of the disorder that confers a beneficial effect on the subject. Methods for measuring cardiac function (e.g., contractile function) are known in the art and are described, for example, in the Textbook of Medical Physiology, Tenth edition, (Guyton et al., W.B. Saunders Co., 2000). For example, cardiac ejection can be monitored using, e.g., echocardiography, nuclear or radiocontrast ventriculography, or magnetic resonance imaging. Other measures of cardiac function include, but are not limited to, myocardial contractility, resting stroke volume, resting heart rate, resting cardiac index, Doppler imaging, cardiovascular performance during stress/exercise. Optionally, cardiac function is increased by at least 5%, 10%, 15%, 20%, 25%, 30%, 40%, 50%, 60%, 70%, 80%, 90%, or even 100% relative to the cardiac function prior to treatment. In some embodiments, the method partially rescues or improves one or more of the following: ejection fraction; left ventricle wall thickness; right ventricle wall thickness; left ventricular wall stress; right ventricular wall stress; ventricular mass; contractile function; cardiac hypertrophy; end diastolic volume; end systolic volume; cardiac output; cardiac index; pulmonary capillary wedge pressure; pulmonary artery pressure; 6 minute walk distance or time, performance on exercise testing, increase in ambulatory activity as monitored remotely by an activity monitor; reduction in serum biomarkers such as N-terminal pro BNP or troponin; and improvement in kidney function as it related to improve blood flow to the kidney.

Treating cardiomyopathy or heart failure in this embodiment would be undertaken to eliminate or postpone need for mechanical support of heart function such as use of a ventricular assist device and/or cardiac transplantation.

All of the U.S. patents, U.S. patent application publications, U.S. patent applications, foreign patents, foreign patent applications and non-patent publications referred to in this specification, are incorporated herein by reference, in their entireties.

From the foregoing it will be appreciated that, although specific embodiments of the invention have been described herein for purposes of illustration, various modifications may be made without deviating from the spirit and scope of the invention.

### EXAMPLES

### Example 1—Materials and Methods

Epigenetic Datasets: For histone ChIP-Seq datasets and ATAC-seq datasets, the “fold change over negative control” bigwig file was used. For transcription factor Chip-seq datasets, peak bed files were used. For Homer computational predictions, a bed file representing the location of the transcription factor motif genome-wide was used. Files were imported into the UCSC genome browser for visualization. When necessary, datasets from mouse cells/tissues or hg38 were overlaid to hg19 using the UCSC liftover tool. For pcHiC data, the CHiCAGO pipeline raw output of three replicates of IPSC-CM promoter capture Hi-C data were downloaded.¹ Probe-probe interactions were filtered. 1 kb was added to both ends of regions interacting with gene promoters. Data from each replicate was intersected using bedtools and retained only genomic interactions that were present in at least two replicates.² Bed files representing pcHi-C interactions were visualized in the UCSC genome browser.

Epigenetic Dataset Downloads and Visualization. Epigenetic datasets were identified from the Encode data repository or GEO. For histone ChIP-Seq datasets and ATAC-seq datasets, the “fold change over negative control” bigwig file was downloaded. For transcription factor Chip-seq datasets, peak bed files were downloaded. For Homer computational predictions, a bed file representing the location of the transcription factor motif genome-wide was downloaded. Files were imported into the UCSC genome browser for visualization. When necessary, datasets from mouse cells/tissues or hg38 were overlaid to hg19 using the UCSC liftover tool.

For pcHiC data, the CHiCAGO pipeline raw output of three replicates of IPSC-CM promoter capture Hi-C data were downloaded¹⁰. Probe-probe interactions were filtered. 1 kb was added to both ends of regions interacting with gene promoters. Data was intersected from each replicate using bedtools and retained only genomic interactions that were present in at least two replicates30. Bed files representing pcHi-C interactions were visualized in the UCSC genome browser.

Enhancer Region Cloning. Candidate enhancer regions were ligated into luciferase plasmids using a Gateway cloning strategy. Candidate enhancer regions were amplified from human genomic DNA using primers with a 5′-CACC overhang using Phusion High-Fidelity DNA polymerase (NEB). An aliquot of the PCR reaction was separated on a 1% agarose-TBE gel to confirm amplification, and the remaining reaction was purified using a PCR Purification Kit (Qiagen). In cases where PCR failed to generate an adequate product, the enhancer region sequence (matching hg19) was synthesized as a dsDNA gGlock gene fragment (IDT). Approximately 5 ng of PCR product or gBlock was ligated into the pENTR/D-TOPO vector following manufacturer's instructions (ThermoFisher). The enhancer region was recombined into pGL4.23-GW (Addgene #60323) using LR Clonase II Enzyme mix (Thermo) with 150 ng of each plasmid. EndoFree Maxipreps (Qiagen) were used to prepare DNA. Plasmids were confirmed using Sanger Sequencing.

Enhancer constructs: Candidate enhancer regions were amplified from human genomic DNA using primers with a 5′-CACC overhang using Phusion High-Fidelity DNA polymerase (NEB). An aliquot of the PCR reaction was separated on a 1% agarose-TBE gel to confirm amplification, and the remaining reaction was purified using a PCR Purification Kit (Qiagen). In cases where PCR failed to generate an adequate product, the enhancer region sequence (matching hg19) was synthesized as a dsDNA gGlock gene fragment (IDT). Approximately 5 ng of PCR product or gBlock was ligated into the pENTR/D-TOPO vector following manufacturer's instructions (ThermoFisher). The enhancer region was recombined into pGL4.23-GW (Addgene #60323) using LR Clonase II Enzyme mix (Thermo) with 150 ng of each plasmid. EndoFree Maxipreps (Qiagen) were used to prepare DNA. Plasmids were confirmed using Sanger Sequencing. In all candidate enhancer plasmids, the enhancer sequence was located 125 bp upstream of the minimal promoter sequence. PCR primers and the genomic regions amplified for each construct are shown in are shown in Table 1.

| Region | | Size | | Distance |
|---|---|---|---|---|
| Name | Primers | (bp) | Coordinates (hg19) | to TSS (bp) |
| MYH7-C1-2 | AGTTCAGCCCCATGAGGTAG | 673 | chr14:23870150-23870823 | 34,047 |
| | GGTACCGAGGCGAGGGATATGGTGAAGG | | | |
| MYH7-C1-3 | GGGTCAGGTCTTTCACAAGC | 698 | chr14:23870761-23871458 | 33,412 |
| | TTTTCCTCCTGTGCCCAAGAC | | | |
| MYH7-C1-4 | TCTTGGGCACAGGAGGAAAATTC | 697 | chr14:23871436-23872136 | 32,734 |
| | TCCCTTCCTCCATTCACCC | | | |
| MYH7-C2 | CTGGCCTTGGCTTTTCTCCAG | 2072 | chr14:23876121-23878188 | 26,682 |
| | CAAACCAGGGTGGCCTCAAG | | | |
| MYH7-C2-1 | AAACCTCCTCTTACCTGGGC | 694 | chr14:23877446-23878141 | 26,729 |
| | TTGGGGAACAGAAGGAGACC | | | |
| MYH7-C2-2 | GCCCTACTCACCTTCCCATTC | 838 | chr14:23876221-23877058 | 27,812 |
| | TGCCTCTCTGCTTCTAACCC | | | |
| MYH7-C2-3 | ACCTGGTTATCCCTTCACGG | 844 | chr14:23876782-23877626 | 27,244 |
| | TGTCACCTCCAGAGCCAAAGG | | | |
| MYH7-C3 | GBLOCK | 961 | chr14:23912000-23912961 | −7,130 |
| MYH7-C4-1 | TGTTCACAATCCCATCCCCA | 1400 | chr14:23913940-23915344 | −9,070 |
| | AGTGGGTCTCTGAAAAGGCA | | | |
| MYH7-C4-2 | TGGCTGGATTCCTGATGTG | 2209 | chr14:23915187-23917391 | −10,317 |
| | CGGACTTTGCCCTTCATAGCACC | | | |
| MYH7-C5 | GCCAGAGGCTGAGCGTGAATTAG | 2223 | chr14:23922666-23924886 | −17,796 |
| | GCAATTTGAATATGATATGCCCAGG | | | |
| MYH7-C5-1 | GBLOCK | 790 | chr14:23923381-23924171 | −18,511 |
| LMNA-C1-1 | CCTGTCCTGGAGTGGCTAAATC | 1156 | chr1:155937201-155938359 | −146,102 |
| | GGGCAGGGGTTAGAATTCCTG | | | |
| LMNA-C1-2 | CATTCGGACTCTCTCTCCCC | 1210 | chr1:155936009-155937220 | −147,241 |
| | TTTAGCCACTCCAGGACAGG | | | |
| LMNA-C2 | GTTAGGTGCCGGGTTTTCTG | 928 | chr14:23904382-23905597 | −17,195 |
| | TGATATGTGCATGTACGGCG | | | |
| LMNA-C3-1 | CTCTCTCGTCCATCCTCCAC | 1108 | chr1:156074366-156075480 | −8,981 |
| | GCTCCTCTTCGGGTCTTGAAAG | | | |
| LMNA-C3-2 | ACTCCTCTAACAGCTGTGGG | 1199 | chr1:156073216-156074415 | −10,046 |
| | CCCCTTGGTGAATGGATCCA | | | |
| LMNA-C4-1 | GAAAGGGATTGGAGCGGAAAG | 1211 | chr1:156092084-156093294 | 7,623 |
| | CAGCAGCCCCTTAACTCTC | | | |
| LMNA-C4-2 | TAACACTGCCACCTTCTGC | 1392 | chr1:156093103-156094494 | 8,642 |
| | TTGGCTAGTCTGTGGGTCTG | | | |
| LMNA-C5 | TGAGATCACCTGGGCGAC | 850 | chr1:156095724-156096574 | 11,263 |
| | AGAAGGGCTGGGCATCCTG | | | |
| LMNA-C6 | CCAGAAAAGGTGAGGGAGGTG | 1101 | chr1:156099538-156100640 | 15,077 |
| | GGGAGGGCCTAGGTAGAAGAG | | | |

Negative distances refer to upstream the transcriptional start site (TSS) and positive distances are downstream.

Luciferase Reporter Assay. HL-1 cardiomyocytes (Millipore Sigma Cat #SCC065) were cultured on fibronectin coated flasks in Claycomb media with 10% HL-1 qualified FBS as previously described.³¹ Twenty-four hours before transfection, 140,000 HL-1 cells per well were plated on to a 12-well plate. On the day of transfection, HL-1 cells were transfected using Lipofecamine 3000 (Thermo Fisher) following manufacturer's instructions. Each well was transfected with 6 μl of 0.15 μM enhancer firefly luciferase plasmid, 50 ng of pRL-SV40 (Promega), 2.5 μl of Lipofecamine3000, and 6 μl of P3000 in 100 μl of Opti-MEM. Cells were allowed to incubate for 6-8 hours, following which half the media was replaced with Claycomb media. Forty-eight hours after transfection, the luciferase assay was performed with the Dual-Glo luciferase assay kit (Promega) according to manufacturer's instructions. The firefly luciferase signal from each well was recorded from three separate replicates and internally normalized to Renilla luciferase signal. Each enhancer construct was tested in a minimum of two separate wells on three separate days.

Induced pluripotent stem cell (iPSC)-derived cardiomyocytes (iPSC-CMs) were generated according to standard protocols³². At approximately day 10 of differentiation, cardiomyocytes were re-plated on to white clear-bottom 96-well plates at 40,000 cells per well. The media was changed every two days and cells began to beat as a syncytium day 14-16. On day 18, cardiomyocytes were transfected with Lipofecamine3000 (Thermo Fisher) according to manufacturer's instructions. Each well was transfected with 0.2 μl of 0.15 μM enhancer firefly luciferase plasmid, 5 ng of pRL-SV40 (Promega), 0.15 μl Lipofecamine3000, and 0.2 μl of P3000 in 10 μl of Opti-MEM. Forty-eight hours after transfection, the luciferase assay was performed with the Dual-Glo luciferase assay kit (Promega) according to manufacturer's instructions. Firefly luciferase signal was read using 96-well plate reader and signals were internally normalized to the same well's Renilla luciferase signal. Each enhancer construct was tested in 8 separate wells on at least three separate cardiomyocyte differentiations.

IPSC Reprogramming, Culturing, and IPSC-CM Differentiation. Human skin fibroblasts were obtained from Coriell (sample name GM03348, 10 year old male) and cultured in DMEM containing 10% FBS. Fibroblasts were re-programmed into induced pluripotent stem cells (IPSCs) via electroporation with pCXLE-hOCT3/4-shp53-F (Addgene plasmid 27077), pCXLE-hSK (Addgene plasmid 27078), and pCXLE-hUL (Addgene plasmid 27080) as described previously³³. IPSCs were maintained on Matrigel-coated 6-well plates with mTeSR-1 (Stem Cell technologies, Cat #85850) and passaged as colonies every 5-7 days using ReLeSR (Stem Cell technologies, Cat #05872).

IPSCs were differentiated into cardiomyocytes (iPSC-CMs) using Wnt modulation as previously described³². Differentiation was conducted in CDM3 (RPMI 1640 with L-glutamine, 213 μg/mL L-asorbic acid 2-phosphate, 500 μg/mL recombinant human albumin)³². Cells were grown to approximately 95% confluency and treated with 6 μM-10 μM CHIR99021 for 24 hours and allowed to recover for 24 hours. Cells were treated with 2 μM Wnt-C59 for 48 hours and then media was changed with CDM3 every two days until beating cardiomyocytes were obtained (approximately day 6-10). In order to prevent cell detachment, beating cardiomyocytes re-plated on to new plates using TrypLE (Thermo Fisher). Media was changed every two days until downstream assays were performed (˜day 20).

CRISPr Enhancer Deletion in IPSCs. To delete enhancer regions, guides targeting the 5′ and 3′ end of enhancer regions were designed using CRISPR³⁴. The guides and primer sequence used in the experiments described herein are provided in Table 2 below.

TABLE 2
Guides and primer sequences

| | | SEQ | |
|---|---|---|---|
| Name | Sequence | ID NOS | Notes |
| MYH7_C3_KO_G1 | GCCTAGAAGTCCGGACACCG | 1 | Guide used to |
| | | | remove C3 |
| | | | Enhancer |
| MYH7_C3_KO_G2 | GTGGTGTGGAACAAAGCGAA | 2 | Guide used to |
| | | | remove C3 |
| | | | Enhancer |
| MYH7_C3_KO_ | CCTAGAAGTCCGGACAACCG | 3 | Guide used to |
| Homo_G1 | | | remove C3 |
| | | | Enhancer in het. |
| | | | IPSCs |
| MYH7_C3_KO_ | TGGTGTGGAACAAAGCCGAA | 4 | Guide used to |
| Homo_G2 | | | remove C3 |
| | | | Enhancer in het. |
| | | | IPSCs |
| MYH7_C4_KO_G1 | ATGGGATTGTGAACAGCGGA | 5 | Guide used to |
| | | | remove C4 |
| | | | Enhancer |
| MYH7_C4_KO_G2 | CGTGATTTGGACTGGCGATC | 6 | Guide used to |
| | | | remove C4 |
| | | | Enhancer |
| MYH7_C6_KO_G1 | CAGAGCCTCCCAAACCCGAA | 7 | Guide used to |
| | | | remove C6 |
| | | | Enhancer |
| MYH7_C6_KO_G2 | TTTGTGGGGAGTGACCGGTC | 8 | Guide used to |
| | | | remove C6 |
| | | | Enhancer |
| MYH7_C6_KO_ | CAGAGCCTOCCAAACCGAA | 9 | Guide used to |
| Homo_G1 | | | remove C4 |
| | | | Enhancer in het. |
| | | | IPSCs |
| MYH7_C3_3Primer | 1.AAGACAGTGGAGTGACGAGG | 10 | Primers used for |
| Genotyping_Mix | 2.AAAGACCTCTAGTGCACCCC | 11 | genotyping C3 |
| | 3.AGAAGAGAACGAAGCGGGAA | 12 | enhancer KO |
| | | | IPSCs |
| MYH7_C4_3Primer | 1.GAGAGGGTGGAGGAGGGT | 13 | Primers used for |
| Genotyping_Mix | 2.TGCATTCCAGGCTGAGTGA | 14 | genotyping C4 |
| | 3.CCCCTTGGTACTGTCCTCAC | 15 | enhancer KO |
| | | | IPSCs |
| MYH7_C6_3Primer | AAAGGGTGCTTGGGACGTAG | 16 | Primers used for |
| Genotyping_Mix | CCTCACTCTCCCCACAAGG | 17 | genotyping C6 |
| | GCCTGAGTAGCCCTGGAAA | 18 | enhancer KO |
| | | | IPSCs |
| hsMYH7_qPCR | F.GCAGCTAAAGGTCAAGGCC | 19 | Gene expression |
| | R.AGCTACTCCTCATTCAAGCC | 20 | in IPSC-CMs |
| | | | Efficiency = 1.04 |
| hsMYH6_qPCR | F.AAGTCCTCCCTCAAGCTCATGGC | 21 | Gene expression |
| | R.ATTTTCCCGGTGGAGAGC | 22 | in IPSC-CMs |
| | | | Efficiency = 0.96 |
| hsTNNT2_qPCR | F.AGGAGACCAGGGCAGAAGATG | 23 | Gene expression |
| | R.CTGGGCTTTGGTTTGGACTCC | 24 | in IPSC-CMs |
| | | | Efficiency = 0.98 |
| hsMYBPC3_qPCR | F.CCCCATCTGAGTACGAGCG | 25 | Gene expression |
| | R.AGCCAGTTCCACGGTCAG | 26 | in IPSC-CMs |
| | | | Efficiency = 0.95 |
| hsSLC8A1_qPCR | F.AGTGCTGGGGAAGATGATGACGACG | 27 | Gene expression |
| | R.AGGATGGAGACAATGAAACACGCCC | 28 | in IPSC-CMs |
| | | | Efficiency = 1.02 |
| hsTNNI3_qPCR | F.CGTGTGGACAAGGTGGATGA | 29 | Gene expression |
| | R.CCGCTTAAACTTGCCTCGAA | 30 | in IPSC-CMs |
| | | | Efficiency = 1.06 |
| hsMYOZ2_qPCR | F.AACACCCCAGATCCACGAAG | 31 | Gene expression |
| | R.GCCTCTAAAAGCTCCGGATC | 32 | in IPSC-CMs |
| | | | Efficiency = 1.02 |
| hsGAPDH_qPCR | F.GTGGACCTGACCTGCCGTCT | 33 | Gene expression |
| | R.GGAGGAGTGGGTGTCGCTGT | 34 | in IPSC-CMs |
| | | | Efficiency = 0.96 |
| MYH7_C3_KO_G1 | GCCTAGAAGTCCGGACACCG | 35 | Guide used to |
| | | | remove C3 |
| | | | Enhancer |
| MYH7_C3_KO_G2 | GTGGTGTGGAACAAAGCGAA | 37 | Guide used to |
| | | | remove C3 |
| | | | Enhancer |
| MYH7_C3_KO_ | CCTAGAAGTCCGGACAACCG | 37 | Guide used to |
| Homo_G1 | | | remove C3 |
| | | | Enhancer in het. |
| | | | IPSCs |
| MYH7_C3_KO_ | TGGTGTGGAACAAAGCCGAA | 38 | Guide used to |
| Homo_G2 | | | remove C3 |
| | | | Enhancer in het. |
| | | | IPSCs |
| MYH7_C4_KO_G1 | ATGGGATTGTGAACAGCGGA | 39 | Guide used to |
| | | | remove C4 |
| | | | Enhancer |
| MYH7_C4_KO_G2 | CGTGATTTGGACTGGCGATC | 40 | Guide used to |
| | | | remove C4 |
| | | | Enhancer |
| MYH7_C6_KO_G1 | CAGAGCCTCCCAAACCCGAA | 41 | Guide used to |
| | | | remove C6 |
| | | | Enhancer |
| MYH7_C6_KO_G2 | TTTGTGGGGAGTGACCGGTC | 42 | Guide used to |
| | | | remove C6 |
| | | | Enhancer |
| MYH7_C6_KO_ | CAGAGCCTCCCAAACCGAA | 43 | Guide used to |
| Homo_G1 | | | remove C4 |
| | | | Enhancer in het. |
| | | | IPSCs |
| MYH7_C3_3Primer | 1.AAGACAGTGGAGTGACGAGG | 44 | Primers used for |
| Genotyping_Mix | 2.AAAGACCTCTAGTGCACCCC | 45 | genotyping C3 |
| | 3.AGAAGAGAACGAAGCGGGAA | 46 | enhancer KO |
| | | | IPSCs |
| MYH7_C4_3Primer | 1.GAGAGGGTGGAGGAGGGT | 47 | Primers used for |
| Genotyping_Mix | 2.TGCATTCCAGGCTGAGTGA | 48 | genotyping C4 |
| | 3.CCCCTTGGTACTGTCCTCAC | 49 | enhancer KO |
| | | | IPSCs |
| MYH7_C6_3Primer | AAAGGGTGCTTGGGACGTAG | 50 | Primers used for |
| Genotyping_Mix | CCTCACTCTCCCCACAAGG | 51 | genotyping C6 |
| | GCCTGAGTAGCCCTGGAAA | 52 | enhancer KO |
| | | | IPSCs |
| hsMYH7_qPCR | F.GCAGCTAAAGGTCAAGGCC | 53 | Gene expression |
| | R.AGCTACTCCTCATTCAAGCC | 54 | in IPSC-CMs |
| | | | Efficiency = 1.04 |
| hsMYH6_qPCR | F.AAGTCCTCCCTCAAGCTCATGGC | 55 | Gene expression |
| | R.ATTTTCCCGGTGGAGAGC | 56 | in IPSC-CMs |
| | | | Efficiency = 0.96 |
| hsTNNT2_qPCR | F.AGGAGACCAGGGCAGAAGATG | 57 | Gene expression |
| | R.CTGGGCTTTGGTTTGGACTCC | 58 | in IPSC-CMs |
| | | | Efficiency = 0.98 |
| hsMYBPC3_qPCR | F.CCCCATCTGAGTACGAGCG | 59 | Gene expression |
| | R.AGCCAGTTCCACGGTCAG | 60 | in IPSC-CMs |
| | | | Efficiency = 0.95 |
| hsSLC8A1_qPCR | F.AGTGCTGGGGAAGATGATGACGACG | 61 | Gene expression |
| | R.AGGATGGAGACAATGAAACACGCCC | 62 | in IPSC-CMs |
| | | | Efficiency = 1.02 |
| hsTNNI3_qPCR | F.CGTGTGGACAAGGTGGATGA | 63 | Gene expression |
| | R.CCGCTTAAACTTGCCTCGAA | 64 | in IPSC-CMs |
| | | | Efficiency = 1.06 |
| hsMYOZ2_qPCR | F.AACACCCCAGATCCACGAAG | 65 | Gene expression |
| | R.GCCTCTAAAAGCTCOGGATC | 66 | in IPSC-CMs |
| | | | Efficiency = 1.02 |
| hsGAPDH_qPCR | F.GTGGACCTGACCTGCCGTCT | 67 | Gene expression |
| | R.GGAGGAGTGGGTGTCGCTGT | 68 | in IPSC-CMs |
| | | | Efficiency = 0.96 |

Guides were ligated into pSpCas9(BB)-2A-Puro (Addgene plasmid #62988) after the U6 promoter using either Bbs1 digestion and ligation or Gibson assembly. DNA preparations of plasmid were prepared using an EndoFree plasmid kit (Qiagen), and plasmid sequences were confirmed with Sanger sequencing. IPSCs were nucleofected using the Neon transfection system (Thermo Fisher). Briefly, GM03348 IPSCs were grown to approximately 70% confluency and treated with mTeSR-1 containing 2 μM thiazovivin (TZV) for one hour. Cells were digested with TrypLE, collected and counted. 3.75 million IPSCs per nucleofection were pelleted at 300 g for 3 min. Cell pellets were resuspended in 125 μl of buffer R and added to an Eppendorf tube containing 1.5 μg or 2.5 μg of each plasmid. Cells were nucleofected in the Neon system in a 100 μl tip with the following settings: 1400 V, 20 ms, 2 pulses. Nucleofected cells were expelled into a single well of Matrigel-coated 6-well plate containing mTeSR-1 supplemented with ClonR (Stem Cell Technologies, Cat #05888) and 2 μM TZV. For each round, a pSpCas9(BB)-2A-GFP (Addgene plasmid #48138) control was included. Twenty-four hours later, cells were treated with mTeSR-1 containing 0.15 μg/mL puromycin. The next day, selection was continued with 0.2 μg/mL puromycin until no viable cells were seen in the GFP control (approximately 2-3 days). Cells were switched to mTeSR-1 supplemented with ClonR and 2 μM TZV and media was changed daily until colonies appeared (5-7 days). Colonies were picked on to 96-well plates, expanded, and split on to two duplicate plates. The first plate was used for cryopreservation in 50% mTeSR-1/ClonR/2 μM TZV and 50% KnockOut Serum replacement/25% DMSO. The second plate was processed for gDNA isolation using the DirectPCR lysis reagent (Viagen, Cat #301-C) following manufacturer's instructions. Colonies were screened for successful enhancer deletion using a 3-primer PCR approach. PCR products were cloned using the TOPO TA cloning kit (Thermo Fisher) and sequenced to determine alleles present. Positive colonies were thawed from the frozen plate, expanded, re-genotyped, and used for differentiation. In cases where no homozygous deletions were obtained, a heterozygous colony was treated with a second round of CRISPR editing.

IPSC Chromosome Analysis and CRIPSr-Off Target Analysis. IPSC Chromosome analysis was conducted using the hPSC genetic analysis kit (Stem Cell Technologies, Cat #07550) following manufacturer's instructions. IPSC lines must show no amplification or deletion in at least 8 of the 9 tested sites to pass our karyotypic quality control standards. The output from the CRISPOR³⁴ guide design tool was used to identify the most likely off target cut sites. Any regions with <3 mismatches and additional off targets that were within or near genes important for cardiac function were selected. Primers were designed to amplify putative off target sites and regions were amplified from gene edited cell gDNA. PCR products were purified using ExoSAp-IT (Thermo) or Ampure XP beads (Beckman Coulter) and sequenced with sanger sequencing. Sanger traces from unedited IPSCs were compared to gene edited lines to identify any off-target changes. Genotype of enhancer deleted cells are shown in Table 2. Off-target analysis is shown in Table 3.

TABLE 2

| | | Genotype | Allele 1 | | Allele 2 | |
|---|---|---|---|---|---|---|
| Target | Clone | Call | Guide 1 Site | Guide 2 Site | Guide 1 Site | Guide 2 Site |
| MYH7 C3 | 1 | Heterozygous | WT +1 (T) | WT +1 (C) | Deletion +0 | Deletion +0 |
| MYH7 C3 | 18 | Homozygous | Deletion +2 (CT) | Deletion +2 (CT) | Deletion −1 (T) | Deletion −1 (T) |
| MYH7 C4 | 2 | Heterozygous | WT +1 (G) | WT +0 | Deletion −6 | Deletion −3 |
| MYH7 C4 | 4 | Homozygous | Deletion +0 | Deletion +1 (G) | Deletion +0 | Deletion −10 |
| MYH7 C6 | 9 | Heterozygous | WT +1 (G) | WT +0 | Deletion −9 | Deletion −10 |
| MYH7 C6 | 2 | Homozygous | Deletion +0 | Deletion +0 | Deletion −9 | Deletion −10 |

Allele changes are shown relative to predicted cut site (+1 means a 1 bp insertion at the predicted cut site).

TABLE 3

| # | Guide | #Mismatches | Location (hg19) | Annotation (Gene) | Result |
|---|---|---|---|---|---|
| 1 | MYH7_C3_KO_G1 | 3 | chr3:43948037- | Intergenic (RP4-672N11.1- | Negative |
| | | | 43948059− | RP4-555D20.3) | |
| 2 | MYH7_C3_KO_G2 | 2 | chr3:8242570- | Intron (LMCD1-AS1) | Negative |
| | | | 8242592:+ | | |
| 3 | MYH7_C3_KO_G2 | 3 | chr2:196514628- | Intron (SLC39A10) | Negative |
| | | | 196514650:− | | |
| 4 | MYH7_C3_KO_G2 | 3 | chr4:25160613- | Exon (SEPSECS) | Negative |
| | | | 25160635:− | | |
| 5 | MYH7_C3_KO_G2 | 4 | chr2:179579180- | Exon (TTN) | Negative |
| | | | 179579202:− | | |
| 6 | MYH7_C4_KO_G1 | 2 | chr2:237534886- | Intergenic (ACKR3- | Negative |
| | | | 237534908:− | AC011286.1) | |
| 7 | MYH7_C4_KO_G1 | 3 | chr18:55971940- | Intron (NEDD4L) | Negative |
| | | | 55971962− | | |
| 8 | MYH7_C4_KO_G1 | 3 | chr1:237019608- | Intron (MTR) | Negative |
| | | | 237019630:+ | | |
| 9 | MYH7_C4_KO_G1 | 4 | chrX:33011884- | Intron (DMD) | Negative |
| | | | 33011906:+ | | |
| 10 | MYH7_C4_KO_G2 | 3 | chr5:37853503- | Intron (GDNF-AS1) | Negative |
| | | | 37853525:+ | | |
| 11 | MYH7_C4_KO_G2 | 3 | chr2:43370103- | Intergenic | Negative |
| | | | 43370125:− | (AC093609.1-THADA) | |
| 12 | MYH7_C6_KO_G1 | 3 | chr2:19143341- | Intergenic | Negative |
| | | | 19143363:+ | (AC106053.1-AC092594.1) | |
| 13 | MYH7_C6_KO_G1 | 3 | chr9:134519754- | Intron (RAPGEF1) | Negative |
| | | | 134519776:− | | |
| 14 | MYH7_C6_KO_G2 | 3 | chr22:18336723- | Intron (MICAL3) | Negative |
| | | | 18336745:+ | | |
| 15 | MYH7_C6_KO_G2 | 4 | chr2:224012528- | Intron (KCNE4) | Negative |
| | | | 224012550+ | | |
| 16 | MYH7_C6_KO_G2 | 4 | chr1:32712994- | Exon (FAM167B) | Negative |
| | | | 32713016:− | | |

IPSC-CM RNA Extraction and qPCR. At ˜day 10 of differentiation, 1 million IPSC-derived cardiomyocytes were plated on a well of 12-well plate. At approximately day 20, cells were washed with PBS and 400 μl of TRIzol (Thermo Fisher) was added directly to the well. Cells were collected into an Eppendorf tube using a cell scraper. Trizol was kept at −80° C. until further processing. Six hundred μl of additional TRIzol was added to the cells and the entire sample was added to a tube containing 250 μl of silica-zirconium beads. Tubes were placed in a bead beater homogenizer (BioSpec) for 1 minute and immediately cooled on ice. Samples were incubated at room temperature for 5 min and then centrifuged at 12,000 g for 5 min to remove unhomogenized cell aggregates. Supernatant was transferred to a new tube and 200 μl of chloroform was added. After vigorous shaking for 30 seconds followed by 10 min incubation with periodic shaking, samples were centrifuged at 12,000 g for 15 min. The upper aqueous layer was added to an equal volume of fresh 70% ethanol and used an input to the Aurum Total RNA Mini Kit (Biorad). RNA was processed according to manufacturer's instructions including on-column DNase digestion. RNA was eluted twice with 30 μl of warmed water and the concentration was measured using a nanodrop spectrophotometer.

The qScript cDNA SuperMix (Quantabio) was used to generate a 100 ng cDNA library. A 1:10 dilution was used as a template in a 3-step SYBR-green qPCR region with a 57° C. annealing temperature. A panel of primers targeting cardiomyocyte references genes (TNNT2, MYBPC3, TNNI3, SLC8A1, MYOZ2 and GAPDH) that passed optimization studies confirming primer specificity and efficiency was used. For enhancer deletion measurements, changes in MYH6 and MYH7 expression were calculated using the delta-delta Cq method using the geometric mean expression of cardiomyocyte reference genes.

SDS-PAGE of Myosin Heavy Chain Isoforms. A 6.25% acrylamide/bis-acrylamide (99:1) resolving gel was prepared by combining 7.5 mL of 25% Acrylamide/bis-acrylamide (99:1), 5.65 mL of 2M Tris pH 8.8, 16.55 mL of ddH20, 300 μl of 10% SDS (w/v), 312 μl 10% ammonium persulfate, and 12.5 μl of TEMED. The resolving gel was allowed to polymerize for 1 hour at room temperature. A 5% acrylamide/bis-acrylamide (99:1) stacking gel was prepared by combining 2 mL of 25% Acrylamide/bis-acrylamide (99:1), 2.5 mL of 0.5M Tris pH 6.8, 5.325 mL of ddH20, 100 μl of 10% SDS (w/v), 90 μl 10% ammonium persulfate, and 6 μl of TEMED. The stacking gel was allowed to polymerize for 8 hours. Lysates of approximately day 20 iPSC-CMs were prepared and protein concentrations were quantified with the Quick-Start Bradford Protein Assay (Bio-Rad). approximately 7 μg of protein was mixed 1:1 with 2× Laemmli Sample Buffer containing β-mercaptoethanol. Samples were loaded into the SDS-polyacrylamide gel described above and separated at 13 mA for 20 min, and 15 mA for 21 hours. After electrophoresis, gels were fixed with a 7% acetic acid/50% methanol solution for 1 hour at room temperature. Protein was visualized with the Sypro Ruby Protein Gel Stain (Thermo Fisher) following manufacturer's instructions. Quantification of band intensities was done using Fiji³⁵.

Engineered Heart Tissue Generation and Measurement of Contractile Properties. Engineered heart tissues (EHTs) were generated according to previously published methods³⁶. iPSC-CMs were differentiated as previously described and when beating cells were present (approximately day 10), cells were washed with PBS and digested with TrypLE (Thermo). One million cells per EHT were centrifuged at 500 g for 5 min and resuspended in 65 μl of EHT media (CDM3³², containing 10% of heat-inactivated FBS, 2 μM thiazovivin, 33 μg/mL aprotinin, and 5 U/mL penicillin/streptomycin), 25 μl of 25 mg/mL fibrinogen and 10 μl of Matrigel (Corning). 100 μl of this EHT mix was added to 3 μl of 100 U/mL thrombin and mixed. The whole mixture was pipetted between PDMS posts (EHT Technologies) in an EHT mold created from 2% agarose and a Teflon spacer in a 24-well Nunc plate (Thermo Fisher). Fibrin gel was allowed to polymerize for 2 hours and then 200 μl of CDM3 was added to the EHT to help detach it from the mold. After 30 min, the PDMS posts were lifted from the mold and the EHT was placed into a new 24 well plate containing 1.6 mL of RPMI containing B27 supplement (Thermo Fisher) and 33 μg/mL aprotinin. Media was changed every other day until further processing. After 20 days of culture, videos of EHT contraction were taken on a KEYENCE BZ-X microscope at 50 fps with 4×4 pixel binning. Videos were imported into Fiji and analyzed with MUSCLEMOTION macro with default settings³⁷. The contraction parameters for each contraction were averaged to give an EHT level measurement.

Flow Cytometry Analysis of IPSC-CM Purity. At approximately day 20 of differentiation, iPSC-CMs were collected using TrypLE (Thermo Fisher). Cells were resuspended in 1 mL of PBS and added to 1 mL of 8% PFA in PBS for fixing. Cells were fixed at 37° C. for 10 min with shaking. Cells were collected by centrifugation at 600 g for 5 min and resuspended in 100 μl ice-cold 90% methanol in PBS per 500,000 starting cells. Cells were stored at −20° C. until further processing. On the day of flow, approximately 1 million cells were aliquoted into two tubes containing 2 mL of 0.5 mg/mL BSA in PBS and pelleted. One tube was resuspended in 100 μl of PBS containing 1:200 dilution of TNNT2-Alexa Fluor 694 (BD Pharmingen #565744) and 1:200 MYBPC3-Alexa Fluor 488 (Santa Cruz Biotechnology #sc-137180 AF488) and the other tube was suspended in PBS alone. Cells were stained for 1 hour at room temperature. Four mL of 0.5 mg/mL BSA in PBS was added to each tube and cells were pelleted. Cells were resuspended in 100 μl in PBS and analyzed on a flow cytometer. The percentage of TNNT2-positive cells was determined by using PBS only as a negative control.

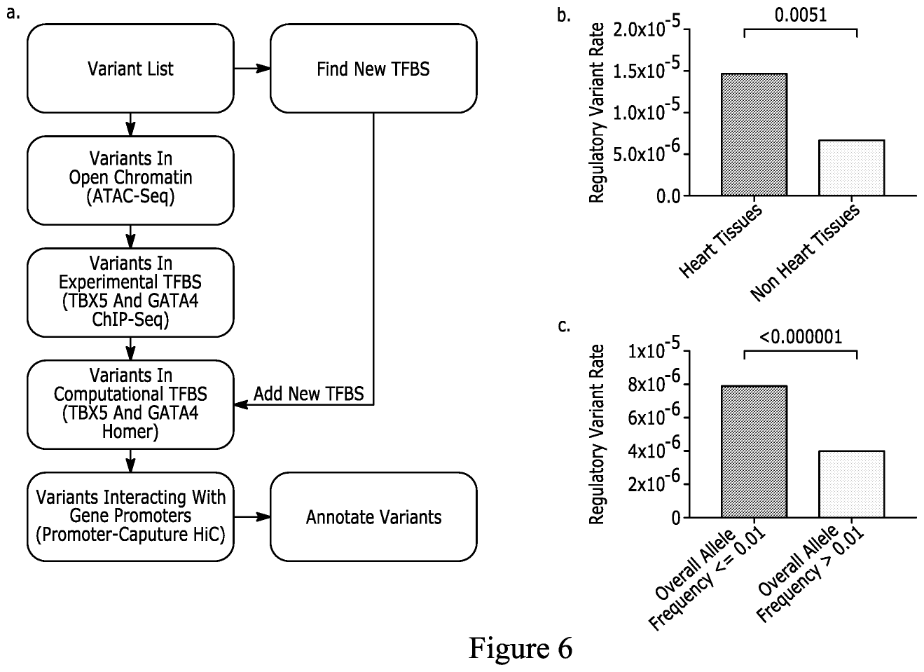

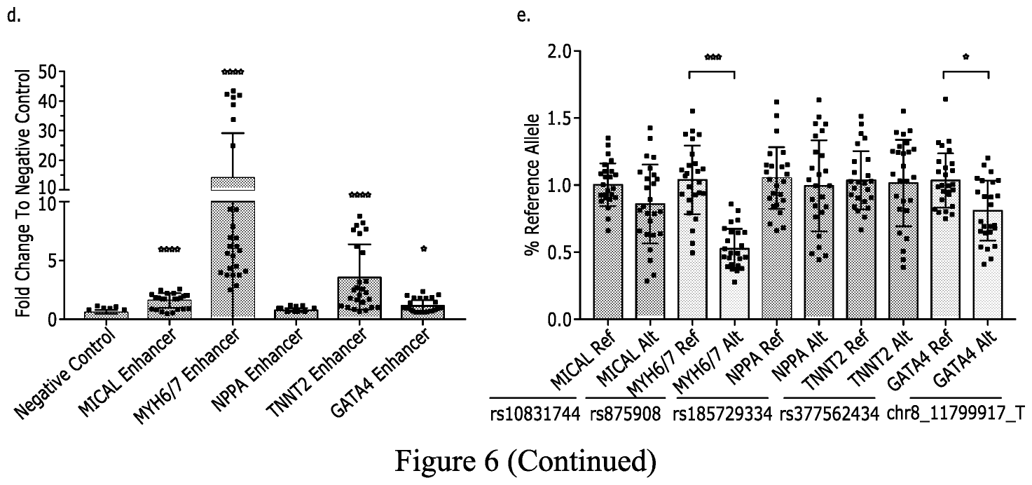

Find Regulatory Variants Computational Pipeline. FIG. 6 shows a schematic of the Find Regulatory Variants computational pipeline. The pipeline relies on the bedtools tool to sequentially filter the starting variant list for variants that overlap regions with epigenetic evidence of enhancer modifying potential³⁰. The epigenetic datasets were all derived from iPSC-CMs and are listed in Table 4.

TABLE 4
Datasets used for epigenomic identification of candidate enhancers

| Target | Dataset | Accession Number | Reference |
|---|---|---|---|
| H3K27Ac Histone | Human LV- ChiP-Seq | ENCSR150QXE | Roadmap Epigenomics |
| Modification | | | Consortium, et al. 2015⁴¹ |
| H3K4me3 Histone | Human LV- ChiP-Seq | ENCFF045RCM | Roadmap Epigenomics |
| Modifications | | | Consortium, et al. 2015⁴¹ |
| Open Chromatin | Human LV-ATAC-Seq | ENCFF148ZMS | ENCODE Project. 2018⁴² |
| | iPSC-CM- ATAC-Seq | GSE85330 | Liu, Q. et al. 2017⁴³ |
| p300 | Human LV- ChiP-Seq | GSE32587 | May, D. et al. 2012⁴⁴ |
| CTCF | Human LV- ChiP-Seq | ENCFF482ZNO | ENCODE Project. 2018⁴² |
| Promoter | iPSC-CM Promoter- | E-MTAB-6014 | Montefiori, L. et al. 2018¹⁰ |
| Interactions | Capture Hi-C | | |
| TAD Boundaries | Human LV- Hi-C | GSE58752 | Leung, D. et al. 2015¹² |
| GATA4 Binding | iPSC-CM-ChIP-Seq | GSM2280004 | Ang, Y. et al. 2016⁴⁵ |
| Sites | HL-1- ChIP-Seq | GSM558904 | He, A. et al. 2011 |
| | Mouse LV- ChIP-Seq | GSM862697 | van den Boogaard, M. et |
| | | | al. 2012⁴⁶ |
| | Computational | HOMER | Heinz, S. et al. 2010³⁹ |
| | Predictions | | |
| TBX5/3 Binding | iPSC-CM-ChIP-Seq | GSM2280011 | Ang, Y. et al. 2016⁴⁵ |
| Sites | HL-1- ChIP-Seq | GSM558908 | He, A. et al. 2011⁴⁷ |
| | Mouse LV- ChIP-Seq | GSM862695 | van den Boogaard, M. et |
| | | | al. 2012⁴⁶ |
| | Computational | HOMER | Heinz, S. et al. 2010³⁹ |
| | Predictions | | |
| NKX2.5 Binding | HL-1- ChIP-Seq | GSM558906 | He, A. et al. 2011⁴⁷ |
| Sites | Mouse LV- ChIP-Seq | GSM862698 | van den Boogaard, M. et |
| | | | al. 2012⁴⁶ |
| | Computational | HOMER | Heinz, S. et al. 2010³⁹ |
| | Predictions | | |
| eRNA Expression | Human LV-CAGE-Seq | GSE147236 | Gacita, A. et al. 2020⁴⁸ |
| Experimentally | Reporter Expression | VISTA Enhancer | Visel, A. Et al. 2007¹³ |
| Validated Heart | in Transgenic Mouse | Browser | |
| Enhancers | Embryos | | |

In datasets where multiple replicates were available, a superset representing all peaks found was created. The pipeline finds variants that are predicted to disrupt or create transcription factor binding sites. In order to use find new transcription factor binding sites created by variants, we used the GATK FastaAlternateReferenceMaker to insert SNP variants into the reference genome³⁸. Homer's scanMotifGenomeWide.pl was then used to search for GATA4 and TBX5 sites in the alternative reference and kept only sites that were new³⁹. In the case of multi-allelic variants, one alternative allele was chosen at random. These additional sites were used in the pipeline alongside sites present in the unchanged reference. This pipeline was executed on variants that passed all quality filters from the gnomAD v.2.1 release.

Association of Enhancer Variant with Phenotypic Data. Phenotypic measurements of heart function and whole genome sequencing data were accessed as in²¹. Individual measures were obtained for left ventricular internal diameter-diastole (LVIDd) and left ventricular posterior wall thickness during diastole (LVPWd) from echocardiogram reports and spanned as much as 14 years of echocardiogram data. The diagnosis of heart failure was determined by ICD9 diagnosis codes 425 and all sub-codes, and ICD10 diagnostic codes 142 and all sub-codes. Trajectory analysis of echo measurements was conducted as in²¹. Briefly, PROC TRAJ in SAS 9.4 was used,⁴⁰ which uses a likelihood function to assign a each individual a phenotypic cluster and probability of belonging to that cluster. An individual's variant status was regressed against cluster probability and was controlled for genetic race (PC1-3) and sex in R.

### Example 2—Integrated Epigenetic Analysis Identifies Candidate Enhancer Regions for MYH7 and LMNA

To find putative modifying regulatory variants associated with cardiomyopathy, the regulatory landscape of two of the most frequently involved genes with this pathology was characterized. Mutations in MYH7 and LMNA are common causes of inherited cardiomyopathy. While both genes have the potential to cause cardiomyopathy, they differ in expression patterns, with MYH7 expression demonstrating tissue restricted expression and LMNA having a broad expression distribution. To identify enhancer regions active in the human left ventricle, multiple datasets including human left ventricle-derived H3K27Ac ChIP-seq and ATAC-seq, as well as ChIP-seq data of genome-wide binding of the cardiogenic transcription factors GATA4, TBX3/5, and NKX2.5 were overlaid from multiple cell/tissue sources (complete list shown in Table 2). Promoter-capture Hi-C data from iPSC-CMs was used to identify genomic regions predicted to interact with promoters¹⁰. Intersection of these datasets identified two enhancer clusters for MYH7 and three for LMNA (FIG. 1). MYH7 cluster 1 overlaps the MYH6 promoter, consistent with their co-regulation in the left ventricle¹¹. MYH7 cluster 2 is approximately 7 kb upstream of MYH7 and is marked by H3K27Ac, CTCF, ATAC signal, transcription factor binding and relatively low H2K4me3 marks. Although many more interactions were identified by promoter capture Hi-C, the integrated analysis highlighted three clusters for LMNA. Cluster 1 was located >100 kb from the LMNA gene within the ARHGEF2 gene, while LMNA cluster 2 was located directly upstream of LMNA. Cluster 3 mapped to the large first intron of LMNA, overlapping the second exon. Similar to the MYH7 sites, the LMNA sites showed H3K27Ac and CTCF marks and open chromatin enrichment. The low H3K4me3 signals differentiated these sites from promoter regions. No enhancer clusters crossed TAD boundaries defined by human left ventricle Hi-C¹².

### Example 3—Candidate Enhancers Display Regulatory Activity in Cardiomyocytes

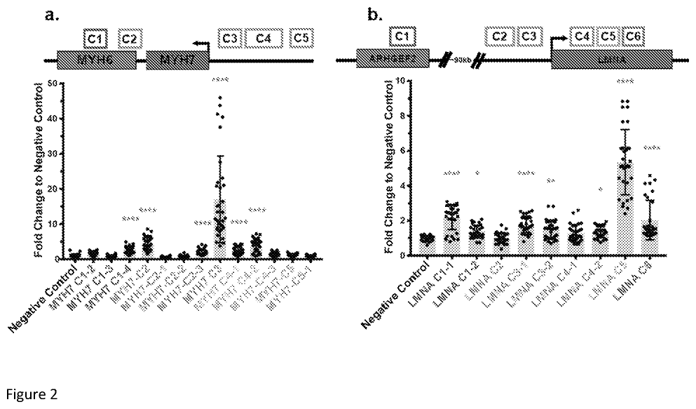

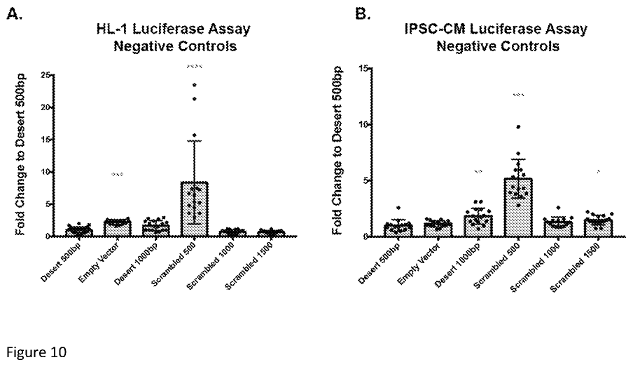

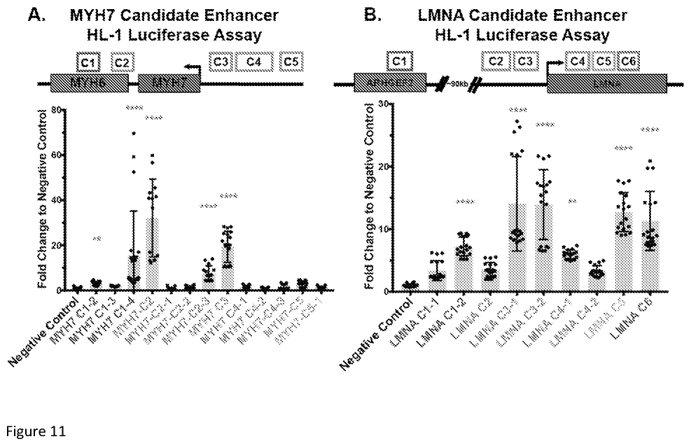

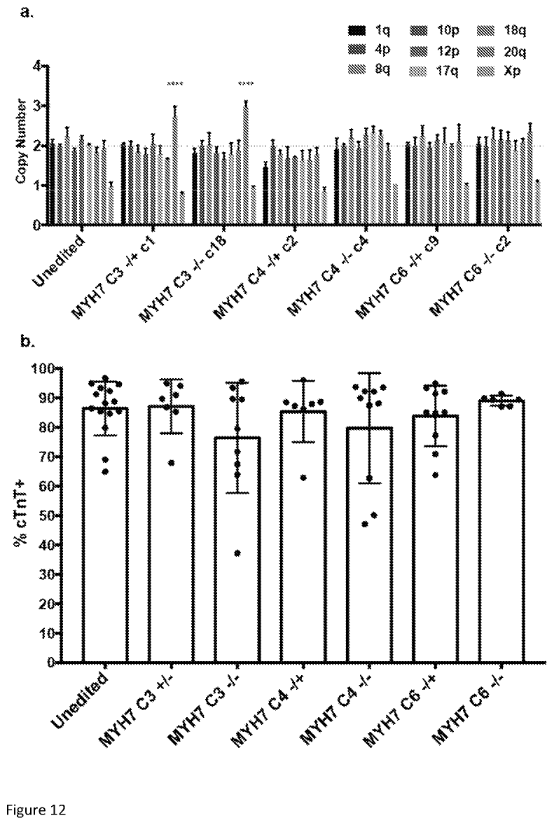

Next, using a luciferase reporter assay, the regulatory potential of the candidate enhancer regions identified in the MYH7 and LMNA loci was determined experimentally. Promoter-capture Hi-C data was used to define the boundaries of individual enhancers within clusters. Because of size, some enhancers were further dissected into smaller regions. Four of five MYH7 enhancer regions tested showed significant activity in iPSC-CMs compared to a negative control genomic desert region (FIG. 2A and FIG. 10). MYH7-C3, which is approximately 7 kb upstream of MYH7 had the strongest signal, consistent with its abundant H2K27Ac ChIP-seq marks. MYH7-C2, which overlaps the MYH6 promoter, was active but with lower magnitude of reporter induction. The MYH7-C2 region also displayed enhancer properties in mouse atrial HL-1 cardiomyocytes, consistent with its role in MYH6 expression in atria (FIG. 11). Searching the VISTA browser revealed that both of these regions also show cardiac enhancer activity in in vivo mouse embryonic reporter assays¹³. MYH7-C4, located further upstream than MYH7-C3, also demonstrated significant enhancer activity in iPSC-CM reporter assays. For LMNA, five of six candidate enhancer regions showed significant activity in iPSC-CMs (FIG. 2B). LMNA enhancer activity was generally lower than MYH7 enhancer activity, consistent with lower LMNA expression in iPSC-CMs. LMNA C5, located at the 3′ end of LMNA's large first intron showed the highest activity. This region showed low H3K4me3 signal, consistent with its role as an enhancer and not a promoter. LMNA C3 showed modest activity iPSC-CMs but does appear as an enhancer in mouse hearts in the VISTA dataset¹³.

### Example 4—Loss of the C3 MYH7 Enhancer Shifts from MYH7 to MYH6, Altering Protein Levels and Increasing Contractile Speed in Engineered Heart Tissues

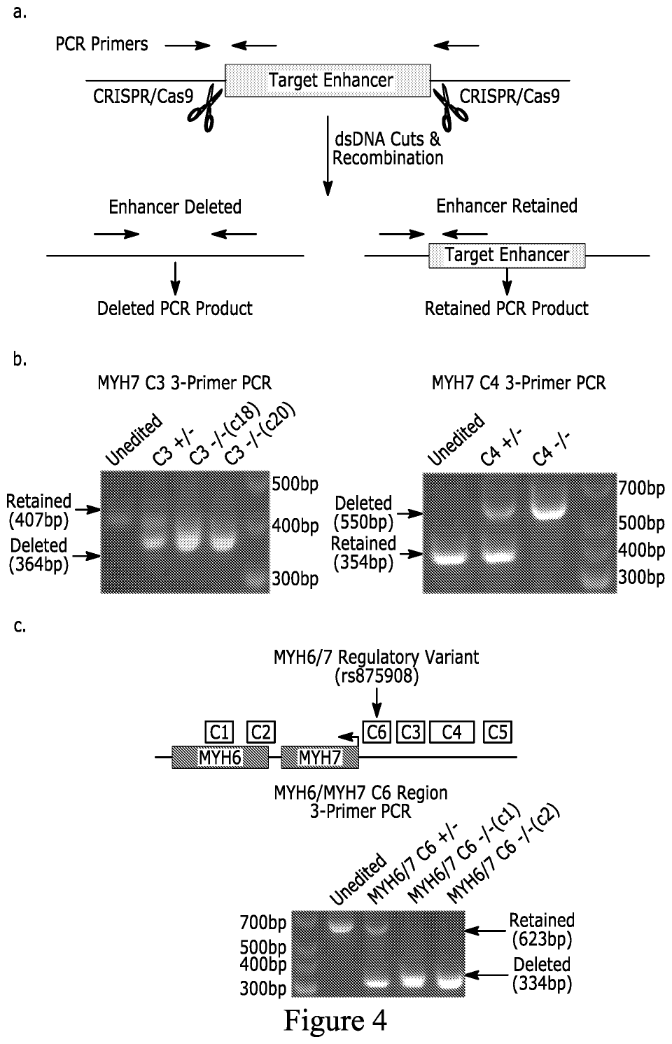

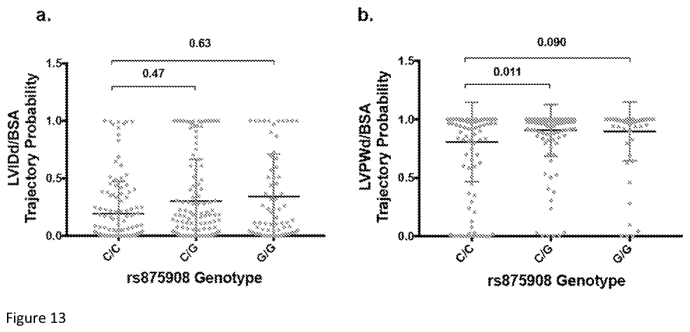

To test if candidate enhancers are required for target gene expression, regions of interest in iPSCs were deleted using gene editing. MYH7-C3 and MYH7-C4 were focused on due to their high activity in reporter assays and intergenic position. LMNA-C3 was not evaluated due to low activity and the potential to disrupt LMNA splicing. A dual cutting CRISPr-Cas9 strategy was employed to remove the candidate enhancer regions (FIG. 4). PCR genotyping confirmed the expected heterozygous and homozygous deletion in independent lines (FIG. 4). All edited cells passed karyotypic and off-target quality control testing (FIG. 13 and Table 3).

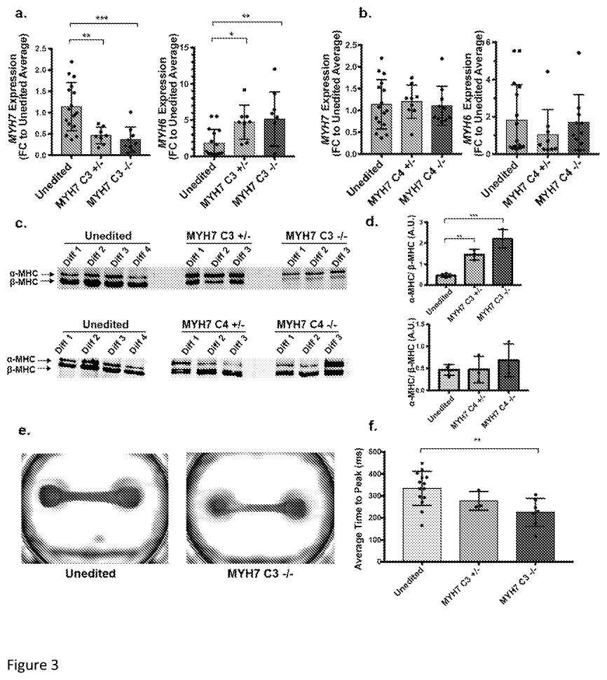

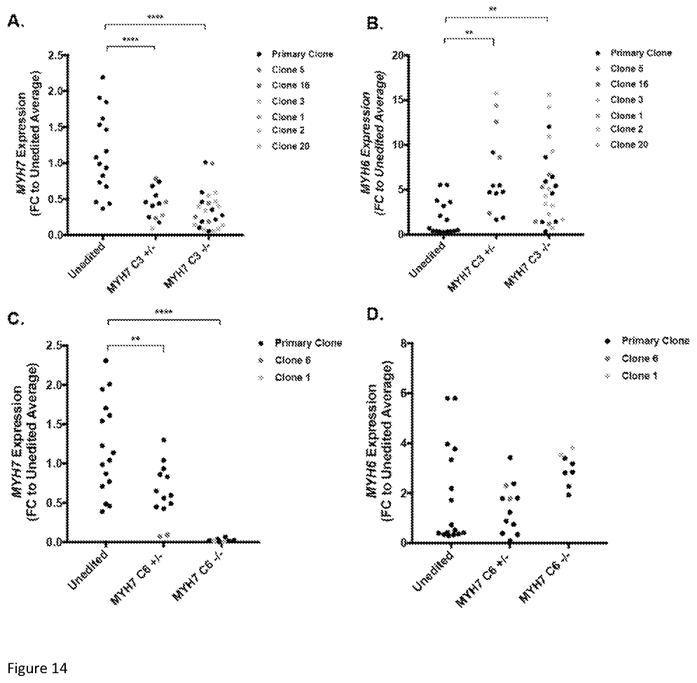

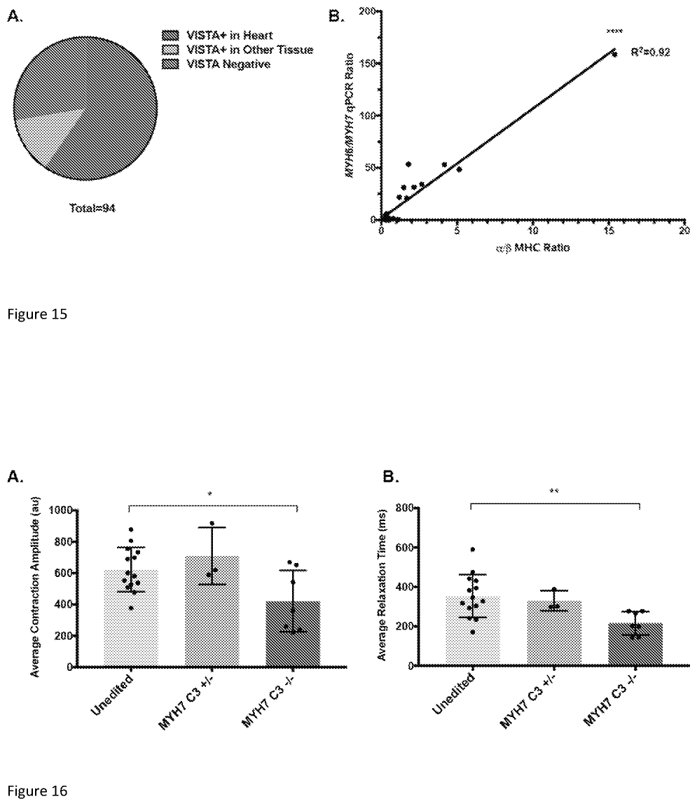

Enhancer-deleted iPSCs were differentiated into cardiomyocytes and measured MYH7 and MYH6 mRNA expression using qPCR. MYH7-C3+/− and −/−cells had a significant decrease in MYH7 expression and increase in MYH6 expression, with dose-dependency (FIG. 3A, FIG. 14). Protein expression was evaluated and it was found MYH7-C3^{+/-} and ^{-/-} IPSC-CMs demonstrated a significant increase in the α-MHC to β-MHC protein ratio (FIG. 3E&F). In general, MYH6 and MYH7 RNA changes were correlated with α-MHC to β-MHC ratio changes in IPSC-CMs (FIG. 15B). Deletion of the MYH7-C4 region had no significant impact on MYH7 or MYH6 mRNA or protein levels, indicating not all upstream regions impact gene and protein expression (FIGS. 3D and E). To ensure comparable maturity and purity, MYH7 and MYH6 gene expression measurements were normalized using a panel of cardiomyocyte genes. Additionally, there were no significant differences between genotypes in IPSC-CM purity as measured by cardiac troponin T (cTNT) flow cytometry (FIG. 4). α-MHC, encoded by MYH6, hydrolyzes ATP at a higher rate than MYH7, which leads to a faster rate of contraction (VanBuren et al., Circ Res. 1995; 77:439-44). The contractile properties of engineered heart tissues (EHTs) generated from MYH7-C3 deleted cardiomyocytes and unedited controls was evaluated. EHTs deleted for MYH7-C3 showed a faster time to peak and shorter relaxation time measurements, consistent with an increased rate of contraction and relaxation (FIG. 3F). Average contraction amplitude was reduced in MYH7-C3 deleted EHTs (FIG. 16), which might reflect a greater energetic cost associated with increased α-MHC expression. Therefore, deletion of MYH7-C3 decreases MYH7 and increases MYH6, which results in a faster contraction rate typical of α-MHC.

### Example 5—Active Cardiac Enhancers Harbor Genetic Variants in Transcription Factor Binding Sites

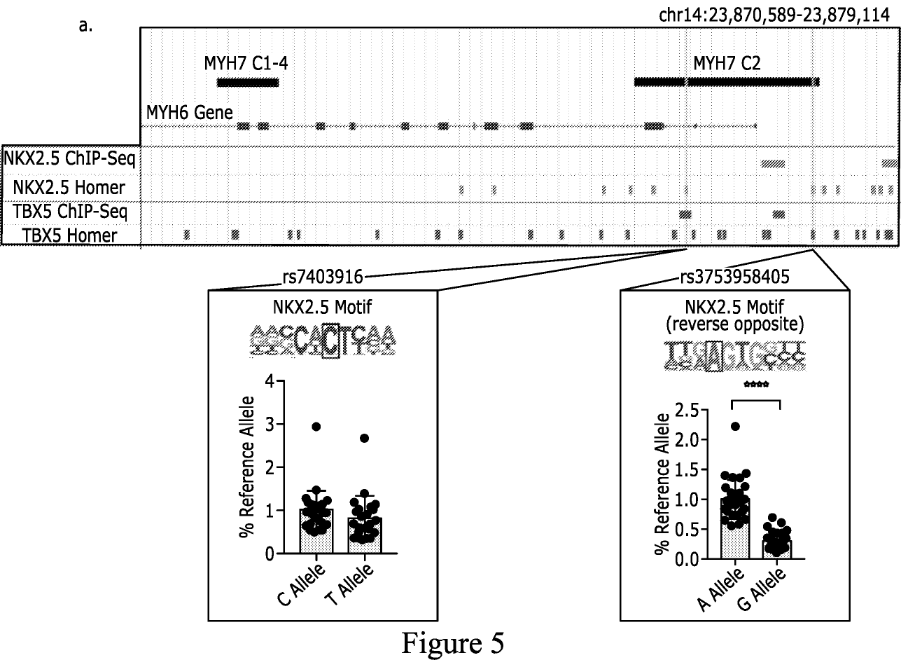

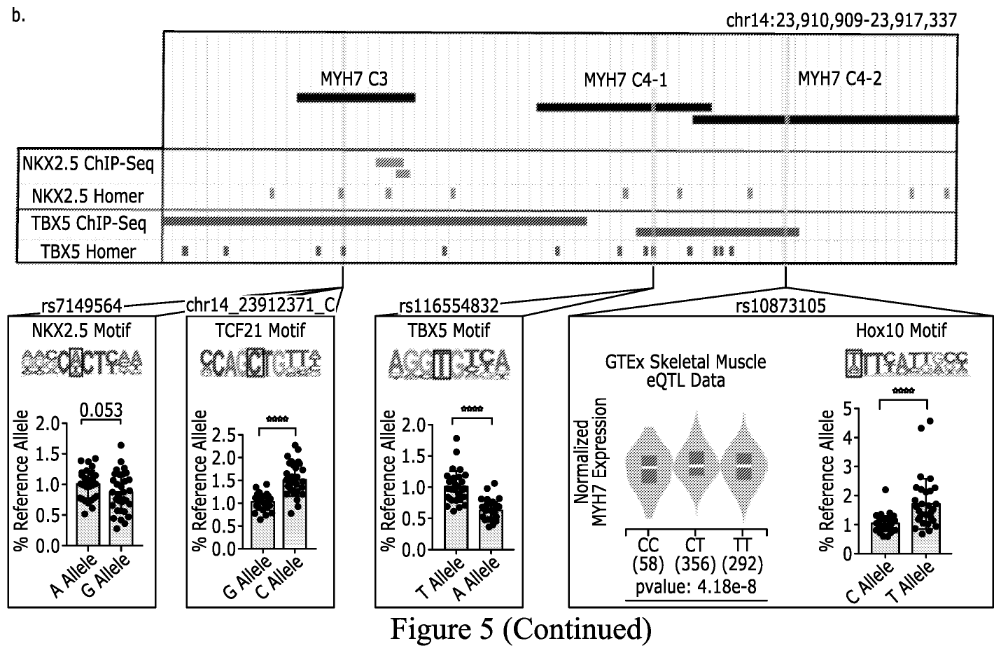

Next, the MYH7 enhancers were characterized for naturally occurring sequence variants using the gnomAD database and those that overlapped cardiac transcription factor binding motifs, and/or were correlated with MYH7 expression in the GTEx eQTL dataset^{15,16} were selected. Six unique variants within MYH7 enhancers that overlapped transcription factor binding motifs and were within or nearby ChIP-seq peaks showing transcription factor binding in cardiac cells were identified (FIGS. 5A&B, top). For each variant, luciferase signals from plasmids carrying the reference or alternative allele in iPSC-CMs were compared. A variant (rs373958405) upstream of MYH6 disrupts a highly conserved site in the NKX2.5 binding motif, and plasmids encoding this variant demonstrated significantly reduced signals in iPSC-CMs compared to the reference allele (FIG. 5A, top right). Within MYH7-C3, rs7149564 was identified which disrupted a less conserved site in the NKX2.5 motif, and consequently, showed a modest trending reduction in luciferase signal (FIG. 5B, bottom left). A nearby variant (chr14_23912371_C), also in MYH7-C3, creates a TCF21 motif and correlated with higher luciferase activity, relative to reference. Within MYH7-C4, a variant (rs116554832) that overlapped a highly conserved site within a TBX5 motif resulted in a reduction in signal (FIG. 5B, bottom middle). A second C4 variant (rs10873105) correlated with MYH7 expression in GTEx skeletal muscle samples. This variant generates a Hox10 motif and causes an increased signal in reporter assays (FIG. 5B, bottom right). These enhancer modifying variants (EMVs) are positioned to regulate cardiac function.

### Example 6—Genome-Wide Evaluation of Enhancer Modifying Variants Identifies Variants Controlling the Activity of Cardiac Enhancers

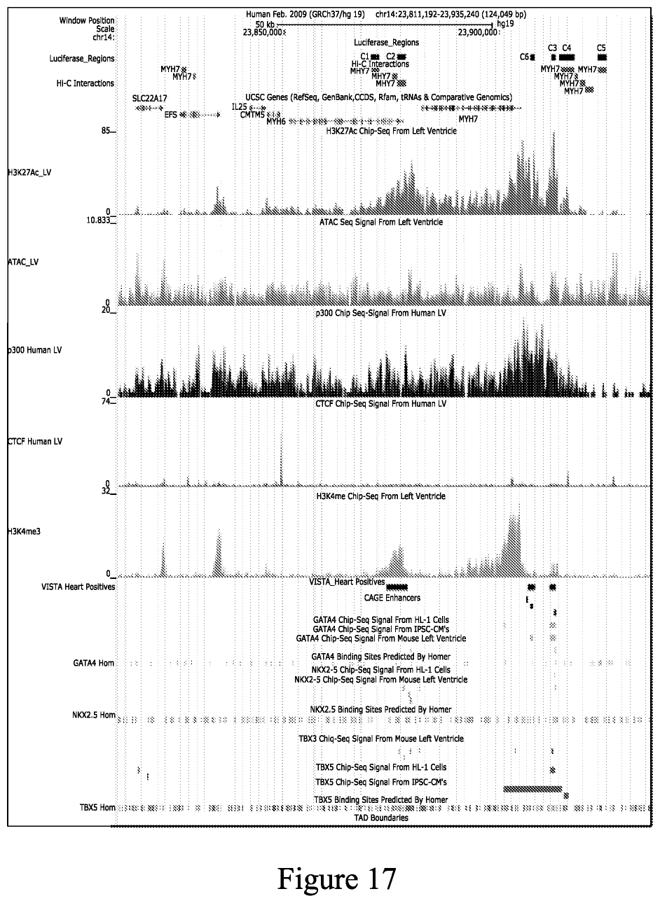

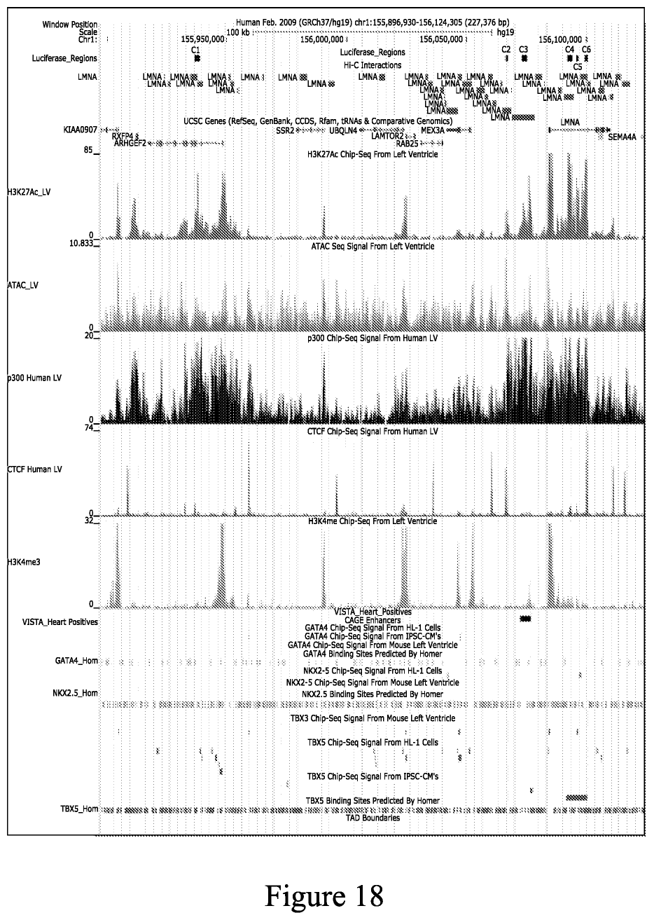

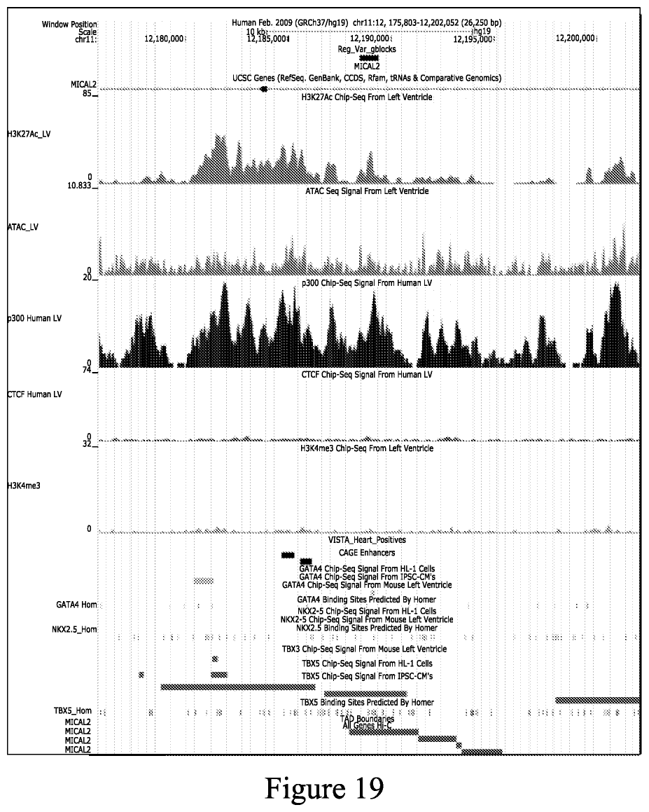

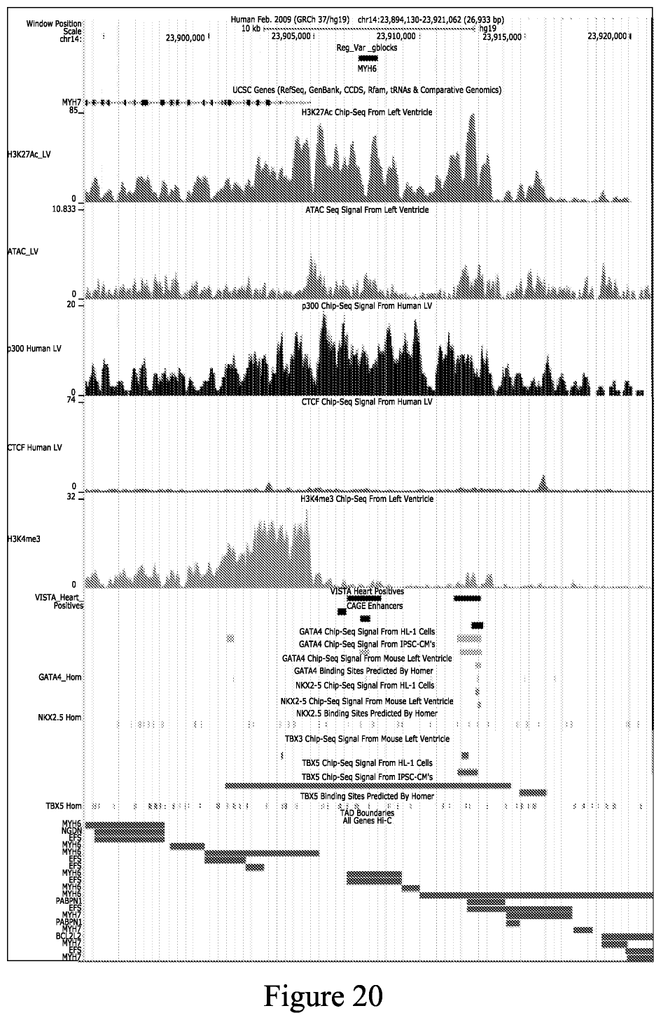

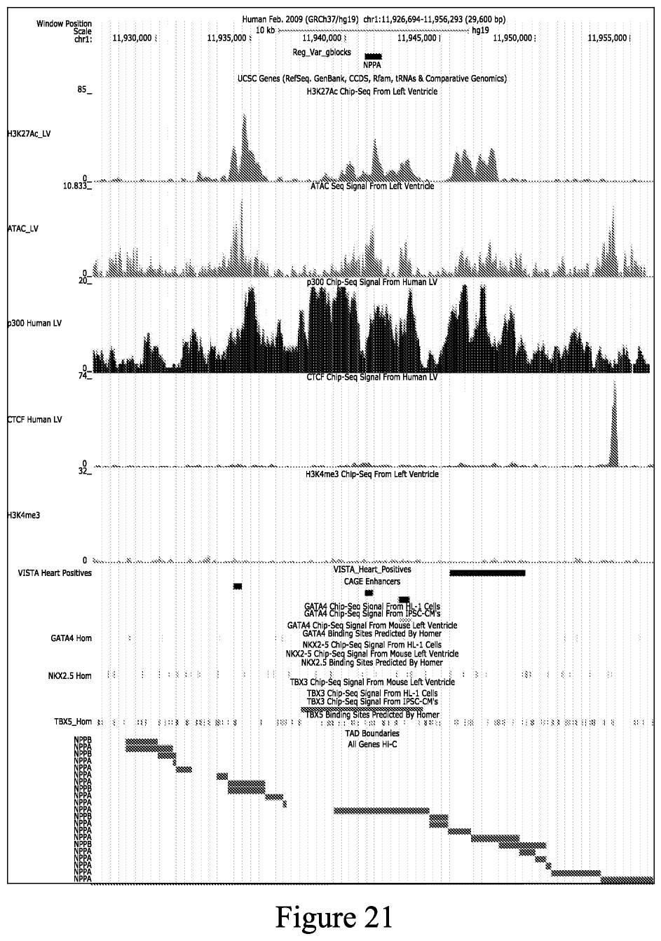

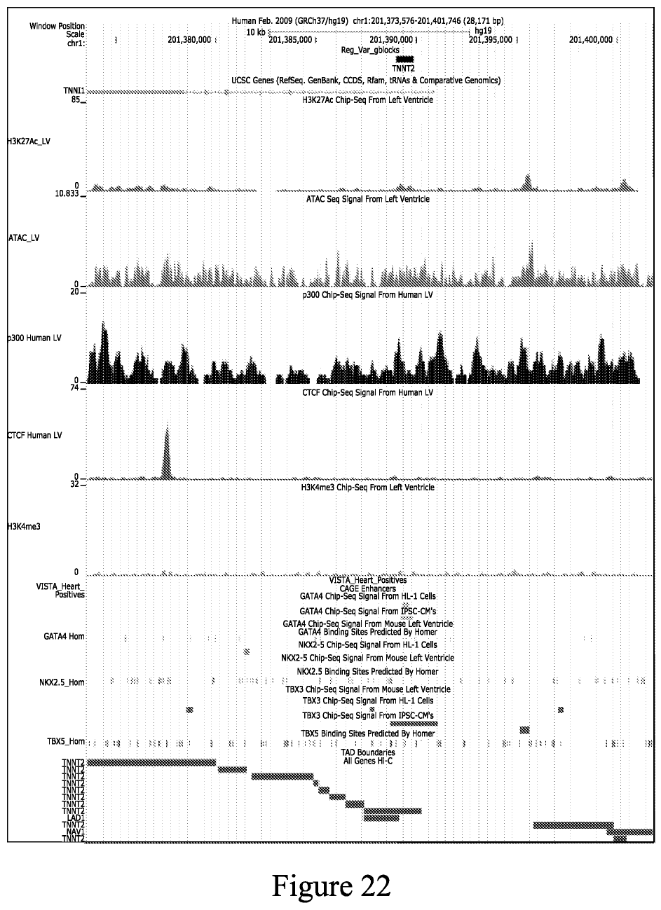

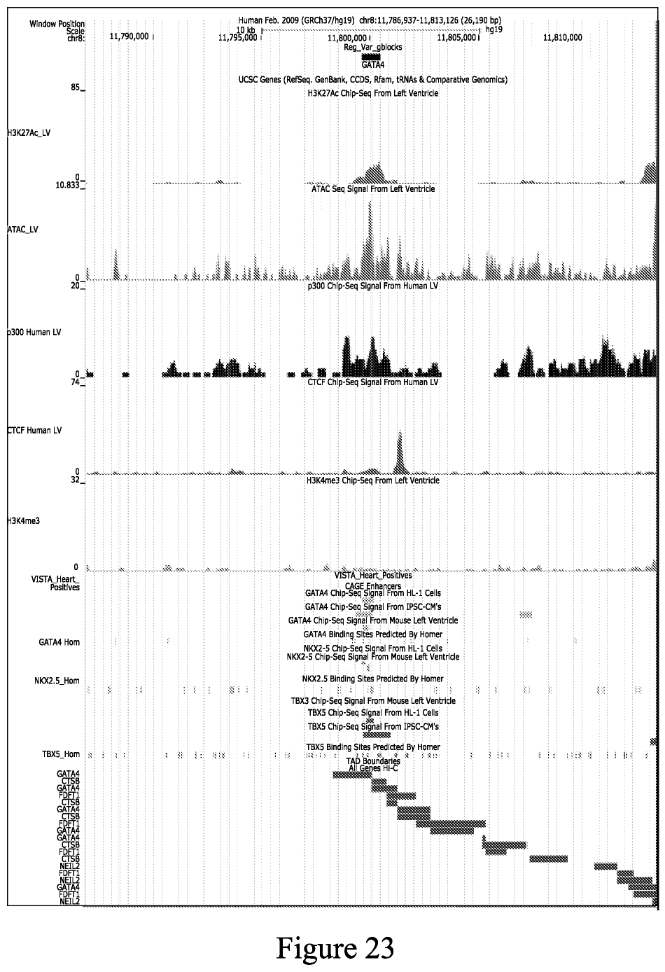

Next, a computational filtering pipeline to use publicly available data from iPSC-CMs to identify variants within enhancer regions that alter transcription factor binding was generated (FIG. 6A). This pipeline was benchmarked using variant sets from GTEx and gnomAD^{15,16}. eQTLs in heart tissues were more likely to be found using this strategy (FIG. 6B). Rare variants were also more likely to survive the filtering steps of this pipeline, consistent with transcription factor binding sites within enhancer regions being under greater constraint and less subject to change (FIG. 6C). This pipeline was executed on gnomAD variants and identified 1,747 variants with EMV potential. Ninety-four of these variants mapped to orthologous regions in the mouse that had been tested in the VISTA database (FIG. 15), and 56 (60%) showed activity in the developing mouse heart. Five variants were selected for experimental testing by choosing variants near five genes important for normal heart function (MICAL2, MYH6, NPPA, TNNT2, and GATA4)¹⁷⁻²⁰. A detailed map of each of the genomic regions is shown in FIGS. 17-23. The candidate enhancers from each of these genes was first tested for luciferase activity in iPSC-CMs, and four of the five variants were active (FIG. 6D). Expression of the reference and alternative alleles in iPSC-CMs was tested. The alternative allele of variants predicted to regulate MYH6 and GATA4 showed significantly reduced function, demonstrating this pipeline has the capacity to identify, on a genome-wide scale, EMVs for cardiac genes.

### Example 7—A Variant ˜2 kb Upstream of MYH7 Correlates with Cardiomyopathic Features in Longitudinal Echocardiographic Imaging

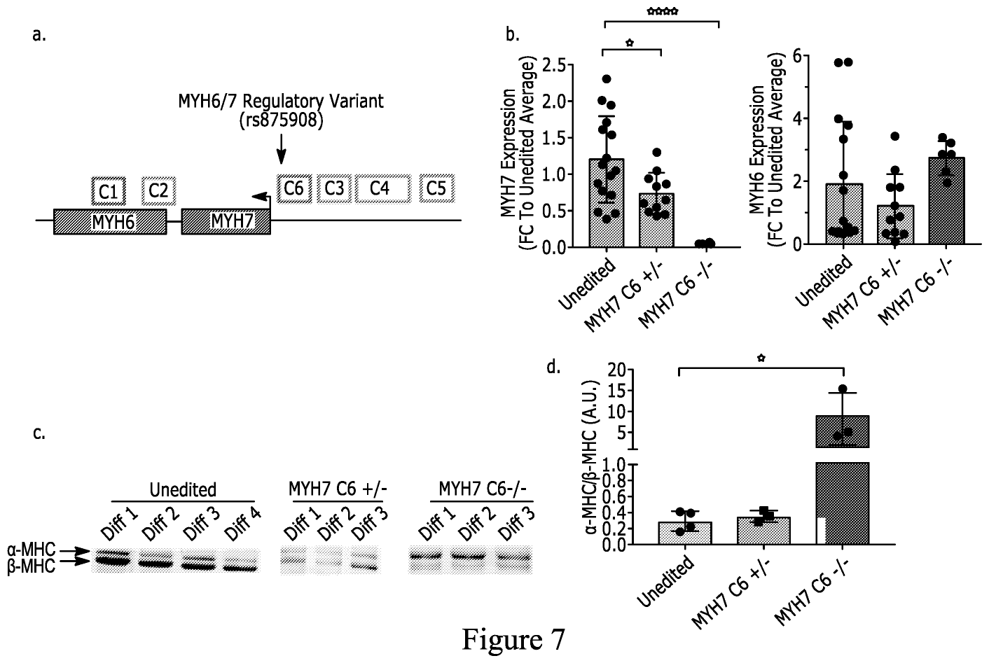

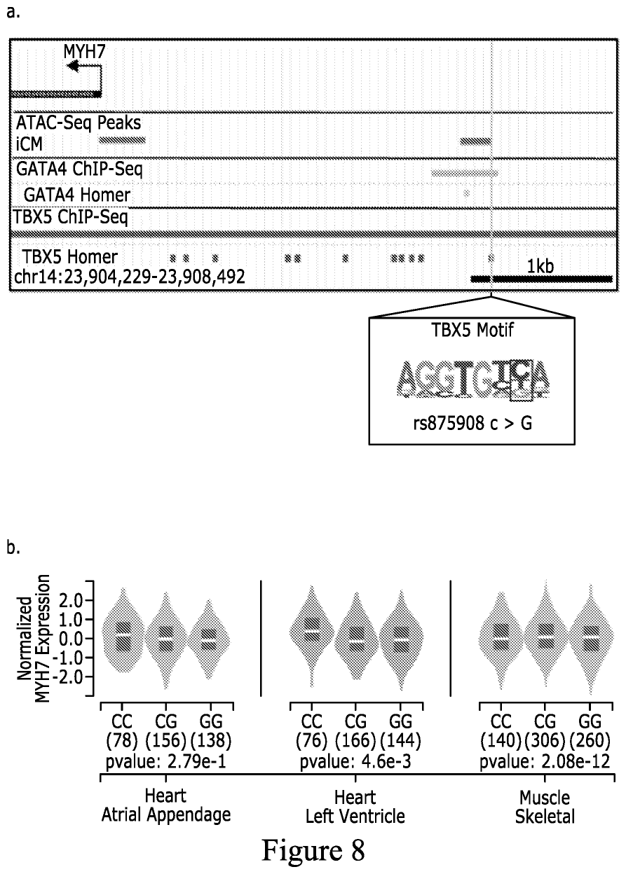

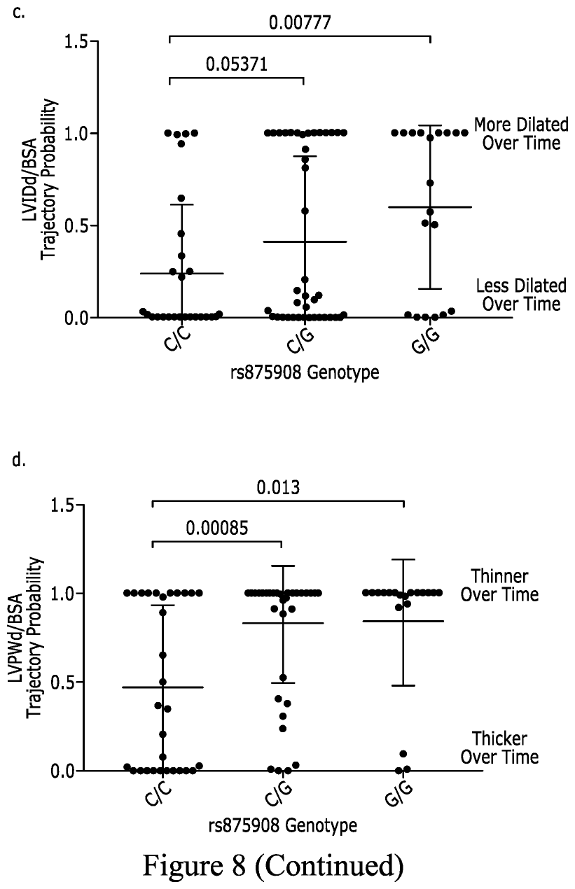

Next, the potential of these identified EMVs to act as modifiers of cardiomyopathy was determined. rs875908, which was predicted to regulate MYH6 by the computational pipeline, is an EMV located approximately 2 kb upstream of MYH7 (FIG. 7A). The region harboring this variant, MYH7-C6, was deleted in iPSCs (FIG. 4). Heterozygous removal of this region in iPSC-CMs caused a reduction in MYH7 expression but no change in MYH6 expression in iPSC-CMs (FIG. 7B). Homozygous deletion of this region showed an approximately 100 fold reduction in MYH7 expression levels and a qualitative, but no significant increase in MYH6 levels (FIG. 7B). Homozygous deleted cells also showed a significant increase in the α/β-MHC protein ratio (FIGS. 7C&D). The rs875908 variant was identified because it is bound by GATA4 and TBX5 and is predicted to disrupt a TBX5 motif (FIG. 8A). GTEx eQTL data show this variant correlates with MYH7 expression in skeletal muscle with trending significance for expression in left ventricle (FIG. 8B).

To ascertain whether rs875908 correlates with cardiac outcomes, we evaluated trajectory probabilities of left ventricular dimensions over time using genomic and echocardiographic information derived from the Northwestern biobank. This approach assigns a probability of maintaining an echocardiographic change overtime²¹. The rs875908-G allele correlates with a more dilated left ventricle over time in participants selected with cardiomyopathy diagnosis codes (FIG. 8C). This correlation was not observed when using clinical data from non-selected biobank participants (FIG. 13). The rs875908-G allele also correlates with a thinner left ventricle posterior wall thickness at end-diastole (LVPWd) over time in those with cardiomyopathy diagnostic codes (FIG. 8B). Variant association with left ventricular wall thickness was also present with all subjects, but with a weaker signal. The cardiomyopathy diagnostic codes in this cohort were for dilated cardiomyopathy. In dilated cardiomyopathy, a thinner wall over time translates to a more diseased heart. The data provided in this Example support that the EMV rs875908 correlated with a more severe dilated cardiomyopathy phenotype and demonstrated the pipeline not only identified EMVs on a genome-wide scale, but that some of these EMVs have clear clinical correlates.

Discussion

Cardiomyopathy gene enhancers. Epigenomic data was integrated to uncover candidate enhancers for a highly expressed and tissue restricted locus like the MYH6/7 genes. As demonstrated herein, this approach can be used on lower and more ubiquitously expressed genes like LMNA, a gene also important for cardiomyopathy. This data integration has the power to identify regulatory regions remote from the gene of interest and uncover human genetic variation that alters the activity of these regions.

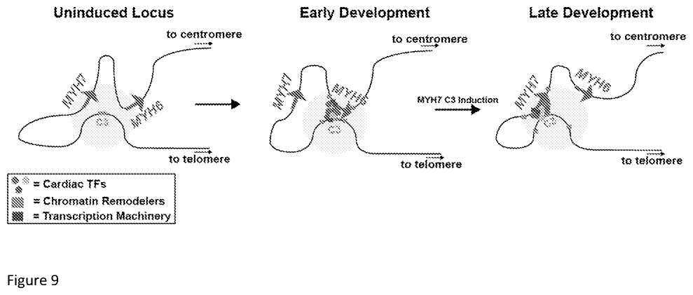

An MYH7/6 Super-Enhancer. Promoter capture Hi-C data from human cardiomyocytes¹⁰ indicates that the MYH7 and MYH6 gene promoters contact each other within 3-dimensional space. Further, an enhancer cluster positioned approximately 7 kb upstream of MYH7 also interacts with the MYH7 gene promoter. Since multiple individual parts of this enhancer cluster have activity in human cardiomyocytes, it is likely this cluster represents a super-enhancer²². Super-enhancers are known to regulate genes critical for cell identity²³. The Examples provided herein demonstrate that deletion of the MYH7-C3 enhancer region reduced MYH7 expression in iPSC-CMs, and, correspondingly, deletion of the MYH7-C3 enhancer increased MYH6 expression resulting in an αMHC/βMHC ratio and a faster rate of contraction in EHTs. Deletion of the MYH7-C3 promoter shifts expression from MYH7 to MYH6, akin to what has been described after thyroid hormone exposure or in the developing ventricle (Metzger et al., Circ Res. 1999; 84:1310-7; Cappelli et al., Circ Res. 1989; 65:446-57; Rundell et al., American journal of physiology Heart and circulatory physiology. 2005; 288:H896-903). The faster rate of contraction/relaxation from deleting MYH7-C3 is distinct from what occurs in hypertrophic cardiomyopathy EHTs, which better reflect the relaxation defects seen in hypertrophic cardiomyopathy (Prondzynski et al., EMBO molecular medicine. 2019; 11:e11115). These data favor a model where the MYH6 and MYH7 promoter regions form a 3-dimensional complex with the super enhancer upstream of MYH7 (FIG. 9). In this model, the super enhancer, containing C3, and additional MYH7-specific enhancer regions induce MYH7 expression, which is critical during heart development, and this same region may be employed in heart failure. The increase in MYH6 expression that was observed may be due to an inhibitory function in C3 or an independent mechanism that compensates for reduced MYH7 expression. These findings are reminiscent of the murine Scn5A-Scn10A locus, a region important for regulating electrical control of the heart²⁴.

Integrated genomics to identify EMVs. The pipeline disclosed herein identified rs875908, a common variant with MAF ranging from 35% to 47% in various populations, as an EMV for cardiomyopathy. This variant correlated with altered MYH7 expression and with a more severe dilated cardiomyopathy phenotype over time, as marked by a more dilated, thinner walled ventricle. The MYH6/7 ratio is known to shift during heart failure, with end stage hearts exhibiting an increase in MYH7 and a decrease in MYH6. With prolonged shift of myosin expression, or a specific magnitude of shift, this change in myosin expression may actually contribute to heart failure²⁵. Supporting this, the MYH6/7 ratio has previously been implicated in heart failure phenotypes²⁶. A distinct contributory mechanism could involve variants within MYH6/7 enhancers, variants in linkage disequilibrium or even pathogenic coding mutations. Varied expression of pathogenic MYH7 mutations has been shown to affect cardiomyopathy phenotypes^{27,28}. A region related to the C6 enhancer, containing the EMV rs875908, was previously deleted in a mouse. Mice missing this C6 orthologous region had reduced MYH7/β-MHC but no change in MYH6/α-MHC²⁹, similar to what was shown here in human cells. This study measured MYH7 expression in the mouse embryonic heart, which differs from the human developing and mature heart. Consistent with the human genetic findings, mouse hearts lacking this enhancer region demonstrated reduced fractional shortening and higher amounts of myofiber disarray, which additionally support the functionality of this region.

A pipeline for EMVs. As deep sequencing data of intergenic regions becomes more available, the importance of noncoding annotation of disease genes will become vital and permit the integration of this information into clinical care. Collectively, the data provided herein provides a robust pipeline to identify genetic variants positioned to alter gene expression. The pipeline disclosed herein identified >1,700 putative EMVs in the gnomAD database, which were linked to multiple genes important for cardiac function like TNNT2, NPPA, GJA5, and MEF2A. Many of the predicted EMVs were infrequent in the population, further supporting the functional role of this type of expression-altering change. However, EMVs were identified at higher population frequency; higher frequency EMVs, because of their prevalence, are more likely to show population level clinical correlates, such as what we could detect using electronic health record data. Targeted assessment of EMVs annotated by specific epigenetic marks can have clinical utility.

### REFERENCES

1. McNally, E. M., Golbus, J. R. & Puckelwartz, M. J. Genetic mutations and mechanisms in dilated cardiomyopathy. J Clin Invest 123, 19-26 (2013).
2. Konno, T., Chang, S., Seidman, J. G. & Seidman, C. E. Genetics of hypertrophic cardiomyopathy. Curr Opin Cardiol 25, 205-9 (2010).
3. Maron, B. J., Rowin, E. J. & Maron, M. S. Global Burden of Hypertrophic Cardiomyopathy. JACC Heart Fail 6, 376-378 (2018).
4. McNally, E. M. & Mestroni, L. Dilated Cardiomyopathy: Genetic Determinants and Mechanisms. Circ Res 121, 731-748 (2017).
5. Marian, A. J. Modifier genes for hypertrophic cardiomyopathy. Curr Opin Cardiol 17, 242-52 (2002).
6. Barp, A. et al. Genetic Modifiers of Duchenne Muscular Dystrophy and Dilated Cardiomyopathy. PLoS One 10, e0141240 (2015).
7. Verdonschot, J. A. J. et al. Mutations in PDLIM5 are rare in dilated cardiomyopathy but are emerging as potential disease modifiers. Mol Genet Genomic Med 8, e1049 (2020).
8. Andersson, R. & Sandelin, A. Determinants of enhancer and promoter activities of regulatory elements. Nat Rev Genet 21, 71-87 (2020).
9. Tanjore, R. et al. Genetic variations of beta-MYH7 in hypertrophic cardiomyopathy and dilated cardiomyopathy. Indian J Hum Genet 16, 67-71 (2010).
10. Montefiori, L. E. et al. A promoter interaction map for cardiovascular disease genetics. Elife 7 (2018).
11. Morkin, E. Regulation of myosin heavy chain genes in the heart. Circulation 87, 1451-60 (1993).
12. Leung, D. et al. Integrative analysis of haplotype-resolved epigenomes across human tissues. Nature 518, 350-354 (2015).
13. Visel, A., Minovitsky, S., Dubchak, I. & Pennacchio, L. A. VISTA Enhancer Browser—a database of tissue-specific human enhancers. Nucleic Acids Res 35, D88-92 (2007).
14. VanBuren, P., Harris, D. E., Alpert, N. R. & Warshaw, D. M. Cardiac V1 and V3 myosins differ in their hydrolytic and mechanical activities in vitro. Circ Res 77, 439-44 (1995).
15. Karczewski, K. J. et al. The mutational constraint spectrum quantified from variation in 141,456 humans. bioRxiv, 531210 (2020).
16. Consortium, G. T. The Genotype-Tissue Expression (GTEx) project. Nat Genet 45, 580-5 (2013).
17. Lundquist, M. R. et al. Redox modification of nuclear actin by MICAL-2 regulates SRF signaling. Cell 156, 563-76 (2014).
18. Houweling, A. C., van Borren, M. M., Moorman, A. F. & Christoffels, V. M. Expression and regulation of the atrial natriuretic factor encoding gene Nppa during development and disease. Cardiovasc Res 67, 583-93 (2005).
19. Hershberger, R. E. et al. Clinical and functional characterization of TNNT2 mutations identified in patients with dilated cardiomyopathy. Circ Cardiovasc Genet 2, 306-13 (2009).
20. Yilbas, A. E. et al. Activation of GATA4 gene expression at the early stage of cardiac specification. Front Chem 2, 12 (2014).
21. Pottinger, T. D. et al. Trajectory analysis of cardiovascular phenotypes from biobank data uncovers novel genetic associations. bioRxiv, 2020.05.10.087130 (2020).
22. Pott, S. & Lieb, J. D. What are super-enhancers? Nat Genet 47, 8-12 (2015).
23. Hnisz, D. et al. Super-enhancers in the control of cell identity and disease. Cell 155, 934-47 (2013).
24. Man, J. C. K. et al. An enhancer cluster controls gene activity and topology of the SCN5A-SCN10A locus in vivo. Nat Commun 10, 4943 (2019).
25. Nakao, K., Minobe, W., Roden, R., Bristow, M. R. & Leinwand, L. A. Myosin heavy chain gene expression in human heart failure. J Clin Invest 100, 2362-70 (1997).
26. Abraham, W. T. et al. Coordinate changes in Myosin heavy chain isoform gene expression are selectively associated with alterations in dilated cardiomyopathy phenotype. Mol Med 8, 750-60 (2002).
27. Tripathi, S. et al. Unequal allelic expression of wild-type and mutated beta-myosin in familial hypertrophic cardiomyopathy. Basic Res Cardiol 106, 1041-55 (2011).
28. Jiang, J., Wakimoto, H., Seidman, J. G. & Seidman, C. E. Allele-specific silencing of mutant Myh6 transcripts in mice suppresses hypertrophic cardiomyopathy. Science 342, 111-4 (2013).
29. Dickel, D. E. et al. Genome-wide compendium and functional assessment of in vivo heart enhancers. Nat Commun 7, 12923 (2016).
30. Quinlan, A. R. & Hall, I. M. BEDTools: a flexible suite of utilities for comparing genomic features. Bioinformatics 26, 841-2 (2010).
31. Claycomb, W. C. et al. HL-1 cells: a cardiac muscle cell line that contracts and retains phenotypic characteristics of the adult cardiomyocyte. Proc Natl Acad Sci USA 95, 2979-84 (1998).
32. Burridge, P. W. et al. Chemically defined generation of human cardiomyocytes. Nat Methods 11, 855-60 (2014).
33. Kim, E. Y. et al. Distinct pathological signatures in human cellular models of myotonic dystrophy subtypes. JCI Insight 4 (2019).
34. Haeussler, M. et al. Evaluation of off-target and on-target scoring algorithms and integration into the guide RNA selection tool CRISPOR. Genome Biol 17, 148 (2016).
35. Schindelin, J. et al. Fiji: an open-source platform for biological-image analysis. Nat Methods 9, 676-82 (2012).
36. Schaaf, S. et al. Generation of strip-format fibrin-based engineered heart tissue (EHT). Methods Mol Biol 1181, 121-9 (2014).
37. Sala, L. et al. MUSCLEMOTION: A Versatile Open Software Tool to Quantify Cardiomyocyte and Cardiac Muscle Contraction In Vitro and In Vivo. Circ Res 122, e5-e16 (2018).
38. McKenna, A. et al. The Genome Analysis Toolkit: a MapReduce framework for analyzing next-generation DNA sequencing data. Genome Res 20, 1297-303 (2010).
39. Heinz, S. et al. Simple combinations of lineage-determining transcription factors prime cis-regulatory elements required for macrophage and B cell identities. Mol Cell 38, 576-89 (2010).
40. Jones, B. L. & Nagin, D. S. Advances in Group-Based Trajectory Modeling and an SAS Procedure for Estimating Them. Sociological Methods & Research 35, 542-571 (2007).
41. Roadmap Epigenomics, C. et al. Integrative analysis of 111 reference human epigenomes. Nature 518, 317-30 (2015).
42. Davis, C. A. et al. The Encyclopedia of DNA elements (ENCODE): data portal update. Nucleic Acids Res 46, D794-D801 (2018).
43. Liu, Q. et al. Genome-Wide Temporal Profiling of Transcriptome and Open Chromatin of Early Cardiomyocyte Differentiation Derived From hiPSCs and hESCs. Circ Res 121, 376-391 (2017).
44. May, D. et al. Large-scale discovery of enhancers from human heart tissue. Nat Genet 44, 89-93 (2011).
45. Ang, Y. S. et al. Disease Model of GATA4 Mutation Reveals Transcription Factor Cooperativity in Human Cardiogenesis. Cell 167, 1734-1749 e22 (2016).
46. van den Boogaard, M. et al. Genetic variation in T-box binding element functionally affects SCN5A/SCN10A enhancer. J Clin Invest 122, 2519-30 (2012).
47. He, A., Kong, S. W., Ma, Q. & Pu, W. T. Co-occupancy by multiple cardiac transcription factors identifies transcriptional enhancers active in heart. Proc Natl Acad Sci USA 108, 5632-7 (2011).
48. Gacita, A. M. et al. Enhancer and promoter usage in the normal and failed human heart. bioRxiv, 2020.03.17.988790 (2020).

What is claimed is:

1. A method for editing the myosin heavy chain 7 (MHY7) gene in a cell comprising introducing into the cell one or more deoxyribonucleic acid (DNA) endonucleases to induce one or more double stranded breaks (DSBs) within chr14:23870150-23924866 as designated in the human genome browser, build 38 (hg38), of the MYH7 gene that results in deletion of an enhancer region of the MYH7 gene.

2. The method of claim 1, wherein the enhancer region is upstream of the MYH7 gene.

3. The method of claim 2, wherein the enhancer region is within the MYH6 gene.

4. The method of claim 3, wherein the enhancer region is MYH7-C1 or MYH7-C2.

5. The method of claim 1, wherein the enhancer region is downstream of the MYH7 gene.

6. The method of claim 5, wherein the enhancer region is MYH7-C6, MYH7-C3, MYH7-C4 or MYH7-C5.

7. The method of claim 6, wherein the enhancer region MYH7-C3 is deleted from the MYH7 gene.

8. The method of any one of claims 1-7, that results in decreased MYH7 expression and increased MYH6 expression in the cell, relative to a cell into which the DNA endonuclease was not introduced.

9. The method of any one of claims 1-8, wherein the one or more DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog thereof.

10. The method of any one of claims 1-9, wherein the method comprises introducing into the cell one or more polynucleotides encoding the one or more DNA endonucleases.

11. The method of any one of claims 1-9, wherein the method comprises introducing into the cell one or more ribonucleic acids (RNAs) encoding the one or more DNA endonucleases.

12. The method of any one of claims 1-11, wherein the method further comprises introducing into the cell one or more guide ribonucleic acids (gRNAs).

13. The method of any one of claims 1-12, wherein the one or more DNA endonucleases is pre-complexed with one or more gRNAs.

14. The method of any one of 1-13, wherein the DNA endonuclease and one or more guide RNAs are delivered by a viral vector.

15. The method of claim 14, wherein the viral vector is a herpes virus vector, an adeno-associated virus (AAV) vector, an adeno virus vector, or a lentiviral vector.

16. The method of claim 15, wherein the viral vector is an adeno-associated virus (AAV) vector.

17. The method of claim 16, wherein the AAV vector is recombinant AAV5, AAV6, AAV8, AAV9, or AAV7.

18. The method of any one of claims 1-17, wherein the one or more guide RNAs (gRNAs) comprise a nucleotide sequence set forth in SEQ ID NOs: 1-68.

19. A method for editing the LMNA gene in a cell by genome editing comprising introducing into the cell one or more deoxyribonucleic acid (DNA) endonucleases to effect one or more double stranded breaks (DSBs) within or near chr1:155937201-156100640 as designated in the human genome browser, build 38 (hg38) of the LMNA gene that results in deletion of one or more enhancer regions of the LMNA gene.

20. The method of claim 19, wherein the one or more enhancer regions is LMNA-C1, LMNA-C2, LMNA-C3, LMNA-C4, LMNA-C5, or LMNA-C6.

21. A method of improving heart function in a subject suffering from cardiomyopathy comprising administering to the subject an agent that both increases myosin heavy chain 6 (MYH6) gene expression and decreases myosin heavy chain 7 (MYH7) gene expression in a cardiac cell of the subject.

22. The method of claim 21, wherein the agent is one or more deoxyribonucleic acid (DNA) endonucleases to effect one or more double stranded breaks (DSBs) within or near enhancer regions of the MYH7 gene of the MYH6 gene that results in deletion of one or more enhancer regions of the MYH7 gene.

23. The method of claim 22, wherein the enhancer region is upstream of the MYH7 gene.

24. The method of claim 22, wherein the enhancer region is within the MYH6 gene.

25. The method of claim 22, wherein the enhancer region is MYH7-C1 or MYH7-C2.

26. The method of claim 22, wherein the enhancer region is downstream of the MYH7 gene.

27. The method of claim 22, wherein the enhancer region is MYH7-C6, MYH7-C3, MYH7-C4 or MYH7-C5.

28. The method of claim 22, wherein the enhancer region MYH7-C3 is deleted from the MYH7 gene.

29. The method of any one of claims 22-28, that results in decreased MYH7 expression and increased MYH6 expression in the cell, relative to a cell into which the DNA endonuclease was not introduced.

30. The method of any one of claims 22-29, wherein the one or more DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog thereof.

31. The method of any one of claims 22-30, wherein the method comprises introducing into the cell one or more polynucleotides encoding the one or more DNA endonucleases.

32. The method of any one of claims 22-31, wherein the method comprises introducing into the cell one or more ribonucleic acids (RNAs) encoding the one or more DNA endonucleases.

33. The method of any one of claims 22-32, wherein the method further comprises introducing into the cell one or more guide ribonucleic acids (gRNAs).

34. The method of any one of claims 22-33, wherein the one or more DNA endonucleases is pre-complexed with one or more gRNAs.

35. The method of any one of 22-34, wherein the DNA endonuclease and one or more guide RNAs are delivered by a viral vector.

36. The method of claim 35, wherein the viral vector is a herpes virus vector, an adeno-associated virus (AAV) vector, an adeno virus vector, or a lentiviral vector.

37. The method of claim 36, wherein the viral vector is an adeno-associated virus (AAV) vector.

38. The method of claim 37, wherein the AAV vector is recombinant AAV5, AAV6, AAV8, AAV9, or AAV7.

39. The method of any one of claims 22-38, wherein the one or more guide RNAs (gRNAs) comprise a nucleotide sequence set forth in SEQ ID NOs: 1-68.

40. A composition comprising one or more guide RNAs (gRNAs) comprise a nucleotide sequence set forth in SEQ ID NOs: 1-68 and a pharmaceutically acceptable carrier, diluent or adjuvant.
